(19) United States
(12) Patent Application Publication
(10) Pub. No.: US 2026/0274851 A1
(43) Pub. Date: Sep. 17, 2026

(54) MODULATORS OF TRPML, THEIR COMPOSITIONS AND METHODS OF USE

(71) Applicant: Caraway Therapeutics, Inc., Rahway, NJ (US)

(72) Inventors: Darby R. Schmidt, Arlington, MA (US); Thomas Wai-Ho Lee, Lexington, MA (US); Rajesh R. Iyengar, Dennis Port, MA (US)

(73) Assignee: Caraway Therapeutics, Inc., Rahway, NJ (US)

(21) Appl. No.: 19/168,086

(22) PCT Filed: Apr. 2, 2024
(86) PCT No.: PCT/US2024/022581

§ 371 Date: Sep. 23, 2025

Related U.S. Application Data
(60) Provisional application No. 63/494,399, filed on Apr. 5, 2023.

(51) Int. Cl. C07D 487/04; A61K 31/519

(52) U.S. Cl. CPC C07D 487/04; A61K 31/519

(57) ABSTRACT

The present disclosure relates to pharmaceutical compounds of the Formulas I or Ia or a pharmaceutically acceptable salt or composition thereof. Also provided are methods of use of TRPML modulators for treating disorders, the modulators including compounds of the Formulas I or Ia. Such methods of use include treatment of ciliopathies.

### CROSS-REFERENCE TO RELATED APPLICATIONS

This application claims the benefit of priority to U.S. Provisional Application No. 63/494,399, filed Apr. 5, 2023, the disclosure of which is incorporated herein by its entirety.

### TECHNICAL FIELD

The present disclosure relates to compounds and compositions which are modulators of TRPML and are useful for treatment of a variety of disorders.

### BACKGROUND

The lysosome is a key organelle that serves as the cell's recycling center. In a highly regulated manner, it breaks down various biomaterials (proteins, lipids, and membranes) into smaller molecules or chemical building blocks, that the cell then employs for energy or as starting materials for new proteins or membranes [see e.g., de Duve, C., The lysosome turns fifty. Nat Cell Biol, 2005. 7(9): p. 847-9. Parkinson-Lawrence, E. J., et al., Lysosomal storage disease: revealing lysosomal function and physiology. Physiology (Bethesda), 2010. 25(2): p. 102-15]. Lysosomal dysfunction due to mutations in the hydrolytic enzyme of lysosomal transport occur in the more than 50 genetically defined Lysosomal Storage Diseases. Interestingly, defects in lysosomal processing can have substantial effects on the function of the organelle beyond the actual enzyme that is mutated—in effect, the system can be gummed up—altering lysosomal degradation and membrane transport/trafficking, creating a positive feedback loop. Because lysosome storage is also seen in common neurodegenerative diseases such as Alzheimer's and Parkinson's, understanding the mechanisms underlying the positive feedback loop may provide therapeutic approaches not only for LSDs, but also for common sporadic neurodegenerative diseases. A lysosome-localized cation channel, TRPML1, has been recently identified as a key regulator of lysosomal function and membrane trafficking processes in the lysosome. Human mutations of TRPML1 cause an inherited lysosomal storage disease, Mucolipidosis IV. This disease is typified by neurodegenerative effects likely driven by the accumulation of lipids and other biomaterials in the cell. The related channels TRPML2 and TRPML3 also regulate lysosomal function.

Many reports suggest that TRPML channel activation is involved in multiple, key lysosomal functions. It can drive the translocation of the Transcription factor (TF)EB to the nucleus. TFEB regulates autophagy and lysosome biogenesis. Overexpression of TFEB has been reported to induce cellular clearance in several lysosome storage diseases, including Pompe Disease, Cystinosis, multiple sulfatase deficiency, as well as common neurodegenerative diseases, including Parkinson's disease and Huntington's disease (Settembre, C., et al., Signals from the lysosome: a control centre for cellular clearance and energy metabolism. Nat Rev Mol Cell Biol, 2013. 14(5): p. 283-96). Therefore, activation of TRPML channels by TRPML agonists may also lead to cellular clearance in all the aforementioned diseases, providing therapeutic targets for these devastating diseases.

Recently, a potent synthetic agonist for TRPML1 has been reported [Shen, D., et al., Lipid storage disorders block lysosomal trafficking by inhibiting a TRP channel and lysosomal calcium release. Nat Commun, 2012. 3: p. 731]. This SF-51-related compound (Mucolipin Synthetic Agonist 1 or ML-SAT) that could induce significant [Ca2+] increases in HEK293 cells stably or transiently expressing TRPML1 protein that has been forced to the plasma membrane via deletion of its lysosomal targeting sequence. High concentrations of ML-SA1 (~10 μM) are needed to effectively activate TRPMLs. Since that concentration is usually difficult to achieve in vivo, ML-SA1 cannot be used to treat the above TRPML related diseases. Liang et al. recently reported a new class of compounds as more potent TRPML activators [WO 2018/005713A1]. These compounds were thought to be useful in treating disorders related to TRPML activities such as lysosome storage diseases, muscular dystrophy, age-related common neurodegenerative diseases, ROS or oxidative stress related diseases, and aging. TRPML activators may also be useful in other disorders.

### SUMMARY OF THE DISCLOSURE

The present disclosure provides for a compound of Formula (I) and pharmaceutically acceptable salts, solvates, hydrates, tautomers, and stereoisomers thereof.

In an aspect, provided is a compound of Formula (I):

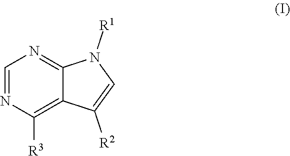

or a pharmaceutically acceptable salt thereof, wherein
R¹ is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b};
R² is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b};
R³ is

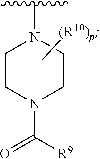

each R¹⁰ is independently selected from the group consisting of C₁₋₆ alkyl and C₁₋₆ haloalkyl, wherein the C₁₋₆ alkyl and C₁₋₆ haloalkyl are optionally substituted with 1-5 substituents independently selected from the group consisting of deuterium, hydroxy, and C₁₋₃ alkoxy; or
two R¹⁰ on the same carbon can be taken together to form oxo; or
any two R¹⁰ can be taken together with the atoms to which they are attached to form a fused ring of 3-7 members, or a bridge of 1 to 3 carbons or a single bond, wherein the ring or bridge is optionally substituted with 1-3 substituents independently selected from the group consisting of deuterium, halogen, hydroxy, C₁₋₆ haloalkyl, and C₁₋₆ alkyl;
R⁹ is C₁₋₆ alkyl substituted with one or more hydroxy, C₁₋₆ alkoxy or C₁₋₆ haloalkoxy and optionally substituted with one or more substituents each independently selected from deuterium, halogen, and C₁₋₃ haloalkyl;
each R^{a} and R^{b} is independently selected from H and C₁₋₆ alkyl; and
p is 0, 1, 2, 3, or 4.

In an aspect, provided is a compound of Formula (Ia):

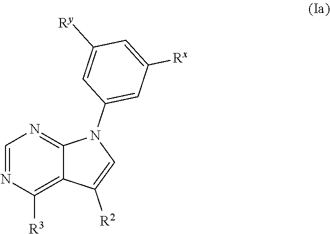

or a pharmaceutically acceptable salt thereof, wherein
R^{x} is selected from the group consisting of halogen, cyano, and C₁₋₆ haloalkoxy;
R^{y} is selected from hydrogen and halogen;
R² is phenyl optionally substituted with one or more C₁₋₆ alkyl or halogen;
R³ is

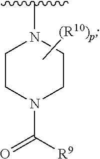

each R¹⁰ is independently selected from C₁₋₆ alkyl and cycloalkyl;
R⁹ is C₁₋₆ alkyl substituted with one or more hydroxy, C₁₋₆ alkoxy or C₁₋₆ haloalkoxy and optionally substituted with one or more substituents each independently selected from deuterium, halogen, and C₁₋₃ haloalkyl; and
p is 0, 1, 2, 3, or 4.

In another aspect, the disclosure provides a method of treating a disease or disorder that can be treated by modulation of TRPML, the method comprising administering to a patient in need thereof a compound described herein or a composition described herein.

Still other objects and advantages of the disclosure will become apparent to those of skill in the art from the disclosure herein, which is simply illustrative and not restrictive. Thus, other embodiments will be recognized by the skilled artisan without departing from the spirit and scope of the disclosure.

### DETAILED DESCRIPTION OF THE DISCLOSURE

As generally described herein, the present disclosure provides compounds (e.g., compounds of Formula (I) or (Ia), or compounds of Table 1, or pharmaceutically acceptable salts thereof) that are useful for disorders (e.g., polycystic kidney disease) associated with modulation of TRPML. “TRPML”, “TRPML ion channel” and “TRPML channel” are used interchangeably throughout.

### Compounds

In an aspect, provided is a compound of Formula (I):

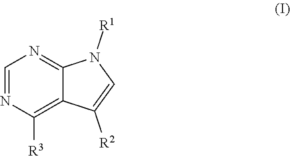

or a pharmaceutically acceptable salt thereof, wherein
R¹ is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b};
R² is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b}; R³ is

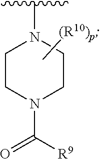

each R¹⁰ is independently selected from the group consisting of C₁₋₆ alkyl, C₁₋₆haloalkyl, and cycloalkyl, wherein the C₁₋₆ alkyl, C₁₋₆ haloalkyl, and cycloalkyl are optionally substituted with 1-5 substituents independently selected from the group consisting of deuterium, hydroxy, and C₁₋₃ alkoxy: or
two R¹⁰ on the same carbon can be taken together to form oxo; or
any two R¹⁰ can be taken together with the atoms to which they are attached to form a fused ring of 3-7 members, or a bridge of 1 to 3 carbons or a single bond, wherein the ring or bridge is optionally substituted with 1-3 substituents independently selected from the group consisting of deuterium, halogen, hydroxy, C₁₋₆ haloalkyl, and C₁₋₆ alkyl;
R⁹ is C₁₋₆ alkyl substituted with one or more hydroxy, C₁₋₆ alkoxy or C₁₋₆ haloalkoxy and optionally substituted with one or more substituents each independently selected from deuterium, halogen, and C₁₋₃ haloalkyl;
each R^{a} and R^{b} is independently selected from H and C₁₋₆ alkyl; and
p is 0, 1, 2, 3, or 4.

In some embodiments, R¹ is 5-10 membered aryl optionally substituted with 1-4 (e.g., 1, 2, 3, or 4) substituents each independently selected from the group consisting of halogen, hydroxy, cyano, C₁₋₆ alkyl. C₁₋₃haloalkyl, C₁₋₆ alkoxy. C₁₋₆ haloalkoxy, C₁₋₆ alkylthio. C₃₋₇ cycloalkyl, and NR^{a}R^{b}.

In some embodiments, R¹ is 5-6 membered aryl optionally substituted with 1-4 (e.g., 1, 2, 3, or 4) substituents each independently selected from the group consisting of halogen, hydroxy, cyano, C₁₋₆ alkyl. C₁₋₃ haloalkyl, C₁₋₆ alkoxy. C₁₋₆ haloalkoxy, C₁₋₆ alkylthio. C₃₋₇ cycloalkyl, and NR^{a}R^{b}.

In some embodiments, R¹ is phenyl optionally substituted with 1-5 (e.g., 1, 2, 3, 4, or 5) substituents. In some embodiments, R¹ is phenyl optionally substituted with 1-4 (e.g., 1, 2, 3, or 4) substituents. In some embodiments. R¹ is phenyl optionally substituted with 1-3 substituents. In some embodiments. R¹ is phenyl optionally substituted with 1 or 2 substituents. The substituents are each independently selected from the group consisting of halogen, hydroxy, cyano, C₁₋₆alkyl, C₁₋₃haloalkyl, C₁₋₆alkoxy, C₁₋₆haloalkoxy, C₁₋₆ alkylthio, C₃₋₇ cycloalkyl, and NR^{a}R^{b}, wherein each R^{a} and R^{b} is independently selected from H and C₁₋₆ alkyl.

In some embodiments, R¹ is phenyl optionally substituted with one or more substituents each independently selected from the group consisting of halogen, hydroxy, cyano. C₁₋₆ alkyl, C₁₋₃ haloalkyl, C₁₋₆ alkoxy, and C₁₋₆ haloalkoxy. In some embodiments, R¹ is phenyl optionally substituted with 1-5 (e.g., 1, 2, 3, 4, or 5) substituents each independently selected from the group consisting of halogen, hydroxy, cyano, C₁₋₆ alkyl, C₁₋₃ haloalkyl, C₁₋₆ alkoxy, and C₁₋₆ haloalkoxy. In some embodiments. R¹ is phenyl optionally substituted with 1-4 (e.g., 1, 2, 3, or 4) substituents each independently selected from the group consisting of halogen, hydroxy, cyano, C₁₋₆ alkyl, C₁₋₃ haloalkyl, C₁₋₆ alkoxy, and C₁₋₆haloalkoxy. In some embodiments, R¹ is phenyl optionally substituted with 1-3 substituents each independently selected from the group consisting of halogen, hydroxy, cyano, C₁₋₆ alkyl, C₁₋₃haloalkyl, C₁₋₆ alkoxy, and C₁₋₆haloalkoxy.

In some embodiments, R¹ is phenyl optionally substituted with one or more (e.g., one, two, three, four, or five) substituents each independently selected from the group consisting of halogen, cyano, and C₁₋₆ haloalkoxy. In some embodiments, R¹ is phenyl optionally substituted with 1-5 (e.g., 1, 2, 3, 4, or 5) substituents each independently selected from the group consisting of halogen, cyano, and C₁₋₆ haloalkoxy. In some embodiments, R¹ is phenyl optionally substituted with 1-4 (e.g., 1, 2, 3, or 4) substituents each independently selected from the group consisting of halogen, cyano, and C₁₋₆ haloalkoxy.

In some embodiments, R¹ is phenyl substituted with cyano.

In some embodiments, R¹ is phenyl substituted with halogen.

In some embodiments, R¹ is phenyl substituted with two halogens.

In some embodiments, R² is 5-10 membered aryl optionally substituted with 1-4 (e.g., 1, 2, 3, or 4) substituents each independently selected from the group consisting of halogen, hydroxy, cyano, C₁₋₆ alkyl. C₁₋₃ haloalkyl, C₁₋₆ alkoxy. C₁₋₆haloalkoxy, C₁₋₆ alkylthio. C₃₋₇ cycloalkyl, and NR^{a}R^{b}.

In some embodiments, R² is 5-6 membered aryl optionally substituted with 1-4 (e.g., 1, 2, 3, or 4) substituents each independently selected from the group consisting of halogen, hydroxy, cyano, C₁₋₆ alkyl, C₁₋₃haloalkyl, C₁₋₆ alkoxy, C₁₋₆haloalkoxy, C₁₋₆ alkylthio. C₃₋₇ cycloalkyl, and NR^{a}R^{b}.

In some embodiments, R² is unsubstituted phenyl.

In some embodiments, R² is substituted phenyl.

In some embodiments, R² is phenyl optionally substituted with 1-5 (e.g., 1, 2, 3, 4, or 5) substituents. In some embodiments, R² is phenyl optionally substituted with 1-4 (e.g., 1, 2, 3, or 4) substituents. In some embodiments, R² is phenyl optionally substituted with 1-3 substituents.

In some embodiments, R² is phenyl optionally substituted with 1 or 2 substituents. The substituents are each independently selected from the group consisting of halogen, hydroxy, cyano, C₁₋₆ alkyl, C₁₋₃haloalkyl, C₁₋₆ alkoxy, C₁₋₆haloalkoxy, C₁₋₆ alkylthio, C₃₋₇ cycloalkyl, and NR^{a}R^{b}, wherein each R^{a} and R^{b} is independently selected from H and C₁₋₆ alkyl.

In some embodiments, R² is phenyl optionally substituted with one or more substituents each independently selected from the group consisting of halogen, hydroxy, cyano. C₁₋₆ alkyl, C₁₋₃ haloalkyl, C₁₋₆ alkoxy, and C₁₋₆ haloalkoxy. In some embodiments, R² is phenyl optionally substituted with 1-5 (e.g., 1, 2, 3, 4, or 5) substituents each independently selected from the haloalkoxy. In some embodiments, R² is phenyl optionally substituted with 1-4 (e.g., 1, 2, 3, or 4) substituents each independently selected from the group consisting of halogen, hydroxy, cyano, C₁₋₆ alkyl, C₁₋₃ haloalkyl. C₁₋₆ alkoxy, and C₁₋₆ haloalkoxy. In some embodiments, R² is phenyl optionally substituted with 1-3 substituents each independently selected from the group consisting of halogen, hydroxy, cyano, C₁₋₆ alkyl, C i-3 haloalkyl, C₁₋₆ alkoxy, and C₁₋₆ haloalkoxy.

In some embodiments, R² is phenyl optionally substituted with one or more (e.g., one, two, three, four, or five) substituents each independently selected from C₁₋₆ alkyl and halogen. In some embodiments, R² is phenyl optionally substituted with 1-5 (e.g. 1, 2, 3, 4, or 5) substituents each independently selected from C₁₋₆ alkyl and halogen. In some embodiments, R² is phenyl optionally substituted with 1-4 (e.g., 1, 2, 3, or 4) substituents each independently selected from C₁₋₆ alkyl and halogen.

In some embodiments, R^{a} and R^{b} are both H. In some embodiments. R^{a} and R^{b} are both C₁₋₆ alkyl. In some embodiments, R^{a} is H, and R^{b} C₁₋₆ alkyl (e.g., methyl, ethyl, propyl, butyl, pentyl, and hexyl).

In some embodiments, p is 0, 1, 2 or 3. In some embodiments, p is 0, 1, or 2. In some embodiments, p is 0 or 1. In some embodiments, p is 0. In some embodiments, p is 1. In some embodiments, p is 2. In some embodiments, p is 3. In some embodiments, p is 4.

In an aspect, provided is a compound of Formula (Ia):

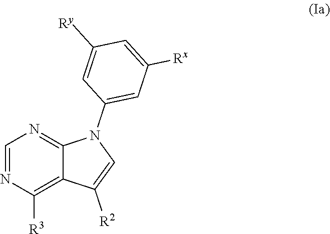

or a pharmaceutically acceptable salt thereof, wherein
R^{x} is selected from the group consisting of halogen, cyano, and C₁₋₆ haloalkoxy;
R^{y} is selected from hydrogen and halogen;
R² is phenyl optionally substituted with one or more C₁₋₆ alkyl or halogen;
R³ is

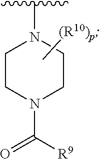

each R¹⁰ is independently selected from C₁₋₆ alkyl and cycloalkyl;
R⁹ is C₁₋₆ alkyl substituted with one or more hydroxy, C₁₋₆ alkoxy or C₁₋₆ haloalkoxy and optionally substituted with one or more substituents each independently selected from deuterium, halogen, and C₁₋₃ haloalkyl; and
p is 0, 1, 2, 3, or 4.

In some embodiments, each R¹⁰ is independently selected from C₁₋₆ cycloalkyl.

In some embodiments, R^{x} is cyano. In some embodiments, R^{x} is fluoro or chloro. In some embodiments, R^{x} is fluoro. In some embodiments, R^{x} is chloro.

In some embodiments, R^{y} is hydrogen. In some embodiments, R^{y} is fluoro.

In some embodiments, R² is unsubstituted phenyl.

In some embodiments, R² is substituted phenyl.

In some embodiments, R² is phenyl optionally substituted with one or more substituents each independently selected from C₁₋₆ alkyl and halogen. In some embodiments, R² is phenyl optionally substituted with 1-5 (e.g., 1, 2, 3, 4, or 5) substituents each independently selected from C₁₋₆ alkyl and halogen. In some embodiments, R² is phenyl optionally substituted with 1-4 (e.g., 1, 2, 3, or 4) substituents each independently selected from C₁₋₆ alkyl and halogen. In some embodiments, R² is phenyl optionally substituted with 1-3 (e.g., 1, 2, or 3) substituents each independently selected from C₁₋₆ alkyl and halogen.

In some embodiments, R² is phenyl optionally substituted with halogen. In some embodiments, R² is phenyl optionally substituted with 1-3 (e.g., 1, 2, or 3) halogen.

In some embodiments, p is 0, 1, 2 or 3. In some embodiments, p is 0, 1, or 2. In some embodiments, p is 0 or 1. In some embodiments, p is 0. In some embodiments, p is 1. In some embodiments, p is 2. In some embodiments, p is 3. In some embodiments, p is 4.

In some embodiments, each R¹⁰ is independently selected from the group consisting of C₁₋₆ alkyl, C₁₋₆ haloalkyl, and 3-7 membered cycloalkyl.

In some embodiments, R¹⁰ is each independently C₁₋₆ alkyl or 3-7 membered cycloalkyl.

In some embodiments, R¹⁰ is each independently 3-7 membered cycloalkyl (e.g., cyclopropyl, cyclobutyl, cyclopentyl, and cyclohexyl)

In some embodiments, R¹⁰ is each independently C₁₋₆ alkyl (e.g., methyl, ethyl, propyl, butyl, pentyl, and hexyl). In some embodiments R¹⁰ is each independently methyl.

In some embodiments, R³ is O R⁹

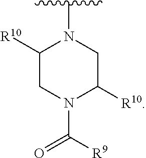

In some embodiments, R³ is

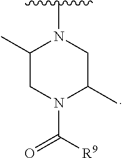

In some embodiments, R³ is

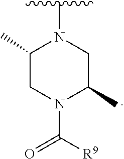

In some embodiments, R⁹ is C₁₋₆ alkyl substituted with 1-5 (e.g., 1, 2, 3, 4, or 5) hydroxy, C₁₋₆ alkoxy or C₁₋₆ haloalkoxy and optionally substituted with 1-5 (e.g., 1, 2, 3, 4, or 5) substituents each independently selected from deuterium, halogen, and C₁₋₃ haloalkyl. In some embodiments, R⁹ is C₁₋₆ alkyl substituted with 1-4 (e.g. 1, 2, 3, or 4) hydroxy, C₁₋₆ alkoxy or C₁₋₆ haloalkoxy and optionally substituted with 1-4 (e.g., 1, 2, 3, or 4) substituents each independently selected from deuterium, halogen, and C₁₋₃ haloalkyl. In some embodiments, R⁹ is C₁₋₆ alkyl substituted with 1-3 (e.g., 1, 2, or 3) hydroxy, C₁₋₆ alkoxy or C₁₋₆ haloalkoxy and optionally substituted with 1-3 (e.g., 1, 2, or 3) substituents each independently selected from deuterium, halogen, and C₁₋₃ haloalkyl.

In some embodiments, R⁹ is C₁₋₆ alkyl substituted with one or more methoxy or hydroxy. In some embodiments, R⁹ is C₁₋₆ alkyl substituted with 1-5 substituents each independently selected from methoxy and hydroxy. In some embodiments, R⁹ is C₁₋₆ alkyl substituted with 1-4 (e.g., 1, 2, 3, or 4) substituents each independently selected from methoxy and hydroxy. In some embodiments. R⁹ is C₁₋₆ alkyl substituted with 1-3 (e.g., 1, 2, or 3) substituents each independently selected from methoxy and hydroxy.

In some embodiments, R⁹ is selected from

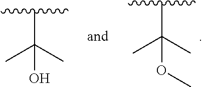

In some embodiments, R⁹ is C₁₋₆ alkyl substituted with one or more ethoxy or trifluoromethylethoxy. In some embodiments, R⁹ is C₁₋₆ alkyl substituted with 1-5 substituents each independently selected from ethoxy or trifluoromethylethoxy. In some embodiments, R⁹ is C₁₋₆ alkyl substituted with 1-4 (e.g., 1, 2, 3, or 4) substituents each independently selected from ethoxy or trifluoromethylethoxy. In some embodiments, R⁹ is C₁₋₆ alkyl substituted with 1-3 (e.g., 1, 2, or 3) substituents each independently selected from ethoxy or trifluoromethylethoxy.

In some embodiments, R⁹ is selected from the group consisting of

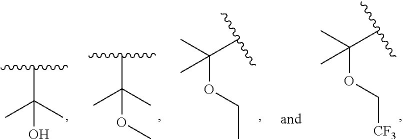

In some embodiments, the compound of Formula (I), or any subformula thereof, is selected from the compounds disclosed in the specification or drawings, or a pharmaceutically acceptable salt thereof.

In some embodiments, the compound achieves at least 50% of the maximal current obtained with 30 μM ML-SA1 in a patch clamp assay for a TRPML and has an EC₅₀ less than 1 μM. In some embodiments, the compound achieves at least 50% of the maximal current obtained with 30 μM ML-SA1 in a patch clamp assay for TRPML1 and has an EC₅₀ less than 1 μM.

In some embodiments, the compound achieves a maximal current obtained with 30 μM ML-SA1 in a patch clamp assay for TRPML1 which is at least 10 fold the maximal current achieved for any other TRPML.

Also provided is a pharmaceutical composition comprising a pharmaceutically acceptable excipient and a compound disclosed herein.

In some embodiments, the
compound is a compound identified in Table 1 below or a pharmaceutically acceptable salt thereof.

TABLE 1
Exemplary compounds

| Cmpd No. | Structure | Cmpd No. | Structure |
|---|---|---|---|
| 101 | 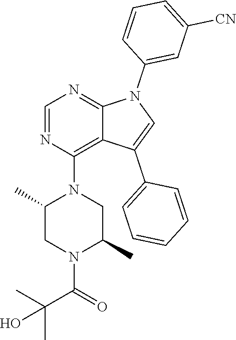 [SMILES: C[C@@H]1CN(c2ncnc3c2c(-c2ccccc2)cn3-c2cccc(C#N)c2)[C@@H](C)CN1C(=O)C(C)(C)O] | 104 | 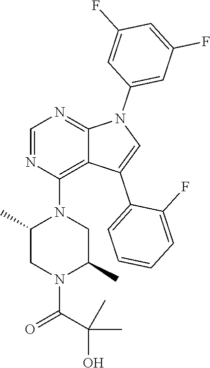 [SMILES: C[C@@H]1CN(c2ncnc3c2c(-c2ccccc2F)cn3-c2cc(F)cc(F)c2)[C@@H](C)CN1C(=O)C(C)(C)O] |
| 102 | 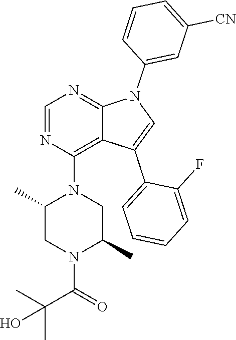 [SMILES: C[C@@H]1CN(c2ncnc3c2c(-c2ccccc2F)cn3-c2cccc(C#N)c2)[C@@H](C)CN1C(=O)C(C)(C)O] | 105 | 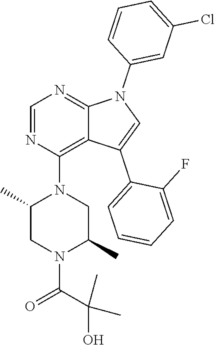 [SMILES: C[C@@H]1CN(c2ncnc3c2c(-c2ccccc2F)cn3-c2cccc(Cl)c2)[C@@H](C)CN1C(=O)C(C)(C)O] |
| 103 | 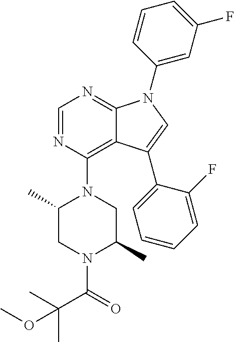 [SMILES: COC(C)(C)C(=O)N1C[C@H](C)N(c2ncnc3c2c(-c2ccccc2F)cn3-c2cccc(F)c2)C[C@H]1C] | | |

In some embodiments, compounds described herein (e.g., a compound of Formula I or Ia) are deuterium-enriched.

Deuterium (D or ²H) is a stable, non-radioactive isotope of hydrogen and has an atomic weight of 2.0144. Hydrogen naturally occurs as a mixture of the isotopes ¹H (hydrogen or protium). D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel over their non-enriched counterparts.

The effects of deuterium modification on a compound's metabolic properties are not predictable, even when deuterium atoms are incorporated at known sites of metabolism. Only by actually preparing and testing a deuterated compound can one determine if and how the rate of metabolism will differ from that of its non-deuterated counterpart. See, for example, Fukuto et al. (J. Med. Chem. 1991, 34, 2871-76). Many compounds have multiple sites where metabolism is possible. The site(s) where deuterium substitution is required and the extent of deuteration necessary to see an effect on metabolism, if any, will be different for each compound.

Unless otherwise stated, when a position is designated specifically as “H” or “hydrogen,” the position is understood to have hydrogen at its natural abundance isotopic composition. Also unless otherwise stated, when a position is designated specifically as “D” or “deuterium,” the position is understood to have deuterium at an abundance that is at least 3000 times greater than the natural abundance of deuterium, which is 0.015% (i.e., the term “D” or “deuterium” indicates at least 45% incorporation of deuterium).

The term “isotopic enrichment factor” as used herein means the ratio between the isotopic abundance of D at the specified position in a compound of this invention and the naturally occurring abundance of that isotope.

Increasing the amount of deuterium present in a compound herein (e.g., a compound of Formula I or Ia) is called “deuterium-enrichment,” and such compounds are referred to as “deuterium-enriched” compounds. If not specifically noted, the percentage of enrichment refers to the percentage of deuterium present in the compound.

In other embodiments, a compound of this invention has an isotopic enrichment factor for each deuterium present at a site designated at a potential site of deuteration on the compound of at least 3500 (52.5% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6466.7 (97% deuterium incorporation), at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

Because the natural abundance of deuterium is about 0.015%, a small percentage of naturally occurring compounds of herein (e.g., a compound of Formula I or Ia) would be expected to have one naturally occurring compound with one deuterium present.

In some embodiments, the compounds herein (e.g., a compound of Formula I or Ia) comprise an amount of deuterium-enrichment that is more than the amount of deuterium-enrichment present in naturally occurring compounds herein (e.g., a compound of Formula I or Ia)

All percentages given for the amount of deuterium present are mole percentages. It can be difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

### Methods of Treatment

Provided herein, in certain embodiments, is a method of modulating TRPML ion channels, the method comprising administering to a patient in need thereof a compound described herein (e.g., a compound of Formula I or Ia) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by modulation of TRPML ion channels, the method comprising administering to a patient in need thereof a compound described herein (e.g., a compound of Formula I or Ia) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML ion channels, the method comprising administering to a patient in need thereof a compound described herein (e.g., a compound of Formula I or Ia) or pharmaceutically acceptable salts, solvates, hydrates, tautomers, stereoisomers, isotopically labeled derivatives thereof, or a composition described herein.

Provided herein, in certain embodiments, is a method of treating a disease or disorder that can be treated by activation of TRPML1, the method comprising administering to a patient in need thereof a compound described herein (e.g., a compound of Formula I or Ia) or pharmaceutically acceptable salts, solvates, hydrates, tautomers, stereoisomers, isotopically labeled derivatives thereof, or a composition described herein.

In addition to compounds of Formula I or Ia, modulators of the TRPML channels have been reported in several publications, including WO2018005713 and WO2018208630, which are incorporated herein in their entirety.

In some embodiments, the TRPML ion channel is TRPML1. In some embodiments, the TRPML ion channel is TRPML2. In some embodiments, the TRPML ion channel is TRPML3.

In some embodiments, the compound is a modulator of TRPML1. In some embodiments, the compound is a modulator of TRPML2. In some embodiments, the compound is a modulator of TRPML3.

In some embodiments, modulation of the TRPML ion channel comprises activation of the ion channel.

In some embodiments, the disease or disorder is a ciliopathy (e.g., polycystic kidney disease). Exemplary ciliopathies include, but not limited to, polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome. Leber's congenital amaurosis, Alstrom syndrome. Jeune asphyxiating thoracic dystrophy, Ellis van Creveld syndrome, Sensenbrenner syndrome, and primary ciliary dyskinesia.

In an aspect, provided is a method of treating a disorder which can be treated by modulation of lysosomes, the method comprising administering to a patient in need thereof a therapeutically effective amount of a pharmaceutical composition of the disclosure or a compound of the disclosure.

In an aspect, provided is a method of treating a disorder selected from the group consisting of a ciliopathy, neurodegenerative disorder, lysosomal storage disorder, lysosomal transport disorder, glycogen storage disorder, cholesteryl ester storage disease, a muscular disease (e.g., muscular dystrophy), a disease related to aging (e.g., photo aging of the skin), macular degeneration (e.g., Stargradt's or age-related), and cancer (e.g., cancers of the blood, brain, bone, lung, liver, kidney, bladder, stomach, breast, prostate, ovary, testes, colon, pancreas, or skin), the method comprising administering to a patient in need thereof a therapeutically effective amount of a pharmaceutical composition of the disclosure or a compound of the disclosure.

In some embodiments, the disorder is a ciliopathy.

In some embodiments, the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy. Ellis van Creveld syndrome. Sensenbrenner syndrome, and primary ciliary dyskinesia.

In some embodiments, the disorder is polycystic kidney disease. In some embodiments, the disorder is autosomal dominant polycystic kidney disease, autosomal recessive polycystic kidney disease, or pancreatic cysts associated with autosomal dominant polycystic kidney disease. In some embodiments, the disorder is autosomal dominant polycystic kidney disease. In some embodiments, the disorder is a neurodegenerative disorder.

In some embodiments, the neurodegenerative disorder is selected from the group consisting of Parkinson's disease, GBA-Parkinson's disease, LRRK2 Parkinson's disease, Huntington's disease, amyotrophic lateral sclerosis (ALS), Alzheimer's disease, progressive supranuclear palsy, frontotemporal dementia, FTDP-17, corticobasal degeneration, Lewy body dementia, Pick's disease, and multi system atrophy.

In some embodiments, the disorder is a lysosomal storage disorder.

In some embodiments, the lysosomal storage disorder is selected from the group consisting of Niemann-Pick disease. Gaucher's disease, neuronopathic Gaucher's disease, sphingolipidoses, Farber disease, Krabbe disease, Galactosialidosis, gangliosidoses, Gaucher Disease, Lysosomal acid lipase deficiency, sulfatidoses, mucopolysaccharidoses, mucolipidoses, lipidoses, and oligosaccharidoses.

In some embodiments, the lysosomal storage disorder is selected from the group consisting of sphingolipidoses, Farber disease, Krabbe disease, Galactosialidosis, Fabry disease, Schindler disease, beta-galactosidase disorder, GM1 gangliosidosis, GM2 gangliosidosis AB variant, GM2 gangliosidosis activator deficiency, Sandhoff disease, Tay-Sachs disease, Gaucher disease, lysosomal acid lipase deficiency, Niemann-Pick disease, metachromatic leukodystrophy, Saposin B deficiency, multiple sulfatase deficiency, Hurler syndrome, Scheie sundrome, Hurler-Scheie syndrome, Hunter syndrome, Sanfilippo syndrome, Morquio syndrome, Maroteaux-Lamy syndrome, Sly syndrome, hyaluronidase deficiency, sialidosis, I-cell disease, pseudo-Hurler polydystrophy, phosphotransferease deficiency, mucolipin 1 deficiency, Santavuori-Haltia disease, Jansky-Bielchowsky disease, Batten-Spielmeyer-Vogt disease, Kufs disease, Finnish variant neuronal ceroid lipfuscinosis, late infantile variant neuronal ceroid lipfuscinosis, type 7 neuronal ceroid lipfuscinosis, northern epilepsy neuronal ceroid lipfuscinosis, Turkish late infantile neuronal ceroid lipfuscinosis. German/Serbian late infantile neuronal ceroid lipfuscinosis, congential cathepsin D deficiency, Wolman disease, alpha-mannosidosis, beta-mannosidosis, aspartylgluosaminuria, and fucosidosis.

In some embodiments, the lysosomal storage disorder is selected from the group consisting of Niemann-Pick disease. Gaucher's disease, and neuronopathic Gaucher's disease.

In some embodiments, the disorder is a lysosomal transport disease selected from the group consisting of cystinosis, pycnodysostosis, Salla disease, sialic acid storage disease, and infantile free sialic acid storage disease.

In some embodiments, the disorder is a glycogen storage disease selected from the group consisting of Pompe disease and Danon disease.

In an aspect, provided is a method of treating a ciliopathy disorder, the method comprising administering to a patient in need thereof a therapeutically effective amount of a compound capable of modulating TRPML, or a therapeutically effective amount of a pharmaceutical composition comprising the compound and a pharmaceutically acceptable excipient.

In some embodiments, the compound is selected from the compounds disclosed in the specification.

In some embodiments, the ciliopathy is selected from the group consisting of polycystic kidney disease, pancreatic cysts in polycystic kidney disease. Bardet-Biedl syndrome, nephronophthisis, Joubert Syndrome, Meckel-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy. Ellis van Creveld syndrome. Sensenbrenner syndrome, and primary ciliary dyskinesia.

In some embodiments, the disorder is polycystic kidney disease.

In some embodiments, the disorder is autosomal dominant polycystic kidney disease, autosomal recessive polycystic kidney disease, or pancreatic cysts associated with autosomal dominant polycystic kidney disease.

In some embodiments, the disorder is autosomal dominant polycystic kidney disease.

In some embodiments, the method further comprises the use of a second therapeutic agent.

In some embodiments, the method is to treat a ciliopathy.

In some embodiments, the second therapeutic agent is selected from the group consisting of an mTOR inhibitor, V2 receptor antagonist, tyrosine kinase inhibitor, somatostatin analog, glucosylceramide synthase inhibitor, microRNA-17 inhibitor, siRNA against p53, KEAP1-Nrf2 activator, xanthine oxidase inhibitor, PPARγ agonist, metformin. and beta hydroxybutyrate.

In some embodiments, the second therapeutic agent is selected from the group consisting of tolvaptan, lixivaptan, mozavaptan, satavaptan, sirolimus, tacrolimus, everolimus, bosutinib, tesavatinib, imatinib, gefitinib, erlotinib, dasatinib, octreotide, pasireotide, venglustat, eliglustat, miglustat, microRNA-17 inhibitor, bardoxolone methyl, allopurinol, oxypurinol, pioglitazone, rosiglitazone, lobeglitazone, metformin, and beta hydroxybutyrate. In some embodiments, the second agent is tolvaptan.

In some embodiments, the second therapeutic agent is selected from the group consisting of an immunomodulator, a calcineurin inhibitor, a renin angiotensin aldosterone system inhibitor, an antiproliferative agent, an alkylating agent, a corticosteroid, an angiotensin converting enzyme inhibitor, an adrenocorticotropic hormone stimulant, an angiotensin receptor blocker, a sodium- glucose transport protein 2 inhibitor, a dual sodium-glucose transport protein ½ inhibitor, a nuclear Factor- 1 (erythroid-derived 2)-like 2 agonist, a chemokine receptor 2 inhibitor, a chemokine receptor 5 inhibitor, an endothelin I receptor antagonist, a beta blocker, a mineralocorticoid receptor antagonist, a loop or thiazide diuretic, a calcium channel blocker, a statin, a short- intermediate or long-acting insulin, a dipeptidyl peptidase 4 inhibitor, a glucagon-like peptide 1 receptor agonist, a sulfonylurea, an apoptosis signal-regulating kinase- 1, a chymase inhibitor, a selective gly cation inhibitor, a renin inhibitor, an interleukin-33 inhibitor, a farnesoid X receptor agonist, a soluble guanylate cyclase stimulator, a thromboxane receptor antagonist, a xanthine oxidase inhibitor, an erythropoietin receptor agonist, a cannabinoid receptor type 1 inverse agonist, a NADPH oxidase inhibitor, an anti-vascular endothelial growth factor B, an anti-fibrotic agent, a neprilysin inhibitor, a dual CD80/CD86 inhibitor, a CD40 antagonist, a cellular cholesterol and lipid blocker, a PDGFR antagonist, a Slit guidance ligand 2, an APOL1 inhibitor, an Nrl2 activator/NF-kB inhibitor, a somatostatin receptor agonist, a PPAR gamma agonist, a AMP activated protein kinase stimulator, a tyrosine kinase inhibitor, a glucosylceramide synthase inhibitor, an arginine vasopressin receptor 2 antagonist, a xanthine oxidase inhibitor, a vasopressin receptor 2 antagonist, anti-amyloid beta antibodies, anti-Tau antibodies, anti-synuclein antibodies, dopamine precursors (e.g., L-DOPA), dopamine agonists (e.g., bromocriptine, cabergoline, pergolide, pramipexole and apomorphine), MAO-B inhibitors (e.g., rasagiline and selegiline), anticholinergics (e.g., orphenadrine, procyclidine and trihexyphenidyl), enhancers of b-glucocerebrosidase activity (e.g., ambroxol and afegostat), amantadine, and agents capable of treating Alzheimer's (e.g., acetylcholinesterase inhibitors such as tacrine, rivastigmine, galantamine, donepezil, and NMDA receptor antagonists such as memantine).

In some embodiments, the second therapeutic agent is selected from the group consisting of COX inhibitors including arylcarboxylic acids (salicylic acid, acetylsalicylic acid, diflunisal, choline magnesium trisalicylate, salicylate, benorylate, flufenamic acid, mefenamic acid, meclofenamic acid and triflumic acid), arylalkanoic acids (diclofenac, fenclofenac, alclofenac, fentiazac, ibuprofen, flurbiprofen, ketoprofen, naproxen, fenoprofen, fenbufen, suprofen, indoprofen, tiaprofenic acid, benoxaprofen, pirprofen, tolmetin, zomepirac, clopinac, indomethacin and sulindac) and enolic acids (phenylbutazone, oxyphenbutazone, azapropazone, feprazone, piroxicam, and isoxicam: treatments for pulmonary hypertension including prostanoids (epoprostenol, iloprost, and treprostinil), endothelin receptor antagonists (bosentan, ambrisentan, and macitentan), phosphodiesterase-5 inhibitors (sildenafil and tadalafil), and sGC stimulators (riociguat); rho-kinase inhibitors, such as Y-27632, fasudil, and H-1 152P; epoprostenol derivatives, such as prostacyclin, treprostinil, beraprost, and iloprost; serotonin blockers, such as sarpogrelate: endothelin receptor antagonists, such as besentan, sitaxsentan, ambrisentan, and TBC3711; PDE inhibitors, such as sildenafil, tadalafil, udenafil, and vardenafil: soluble gunaylate cyclase inhibitors such as riociguat and vericiguat; calcium channel blockers, such as amlodipine, bepridil, clentiazem, diltiazem, fendiline, gallopamil, mibefradil, prenylamine, semotiadil, terodiline, verapamil, aranidipine, bamidipine, benidipine, cilnidipine, efonidipine, elgodipine, felodipine, isradipine, lacidipine, lercanidipine, manidipine, nicardipine, nifedipine, nilvadipine, nimodipine, nisoldipine, nitrendipine, cinnarizine, flunarizine, lidoflazine, lomerizine, bencyclane, etafenone, and perhexiline; tyrosine kinase inhibitors, such as imatinib; inhaled nitric oxide and nitric oxide-donating agents, such as inhaled nitrite; IκB inhibitors, such as IMD 1041; prostacyclin receptor agonists, such as selexipag; stimulators of hematopoiesis, such as TXA 127 (angiotensin (1-7)), darbepoetin alfa, erythropoetin, and epoetin alfa; anticoagulants and platelet-inhibiting agents; and diuretics; dietary and nutritional supplements such as acetyl-L-carnitine, octacosanol, evening primrose oil, vitamin B6, tyrosine, phenylalanine, vitamin C, L-dopa: immunosuppressants (for transplants and autoimmune-related RKD); anti-hypertensive drugs (for high blood pressure-related RKD, e.g., angiotensin-converting enzyme inhibitors and angiotensin receptor blockers); insulin (for diabetic RKD): lipid/cholesterol-lowering agents (e.g., HMG-CoA reductase inhibitors such as atorvastatin or simvastatin); and treatments for hyperphosphatemia or hyperparathyroidism associated with CKD (e.g., sevelamer acetate, cinacalcet).

The present disclosure further provides pharmaceutical compositions comprising a compound provided herein, or a pharmaceutically acceptable salt thereof, and at least one pharmaceutically acceptable carrier. The present disclosure further provides methods of modulating TRPML in a subject, the method comprising administering to the subject a therapeutically effective amount of a compound provided herein, or a pharmaceutically acceptable salt thereof.

The present disclosure further provides a method of treating a disease or disorder in a subject, the method comprising:
(a) detecting a disease or disorder associated with TRPML; and
(b) administering to the subject a therapeutically effective amount of a compound provided herein, or a pharmaceutically acceptable salt thereof.

In certain embodiments, exemplary compounds of Formula I or La, include the compounds described in Table 1 and in the Examples, as well as pharmaceutically acceptable salts, solvates, hydrates, tautomers, and stereoisomers thereof.

Accordingly, the present disclosure provides compounds useful for treating ciliopathies and related diseases.

Compounds that modulate TRPML channels may be useful in the prophylaxis and treatment of any of the foregoing injuries, diseases, disorders, or conditions. In addition to in vitro assays of the activity of these compounds, their efficacy can be readily tested in one or more animal models.

This disclosure is not limited in its application to the details of the methods and compositions described herein. Also, the phraseology and terminology used herein is for the purpose of description and should not be regarded as limiting.

### Pharmaceutical Compositions and Routes of Administration

The present disclosure provides pharmaceutical compositions comprising a compound provided herein, or a pharmaceutically acceptable salt thereof, and at least one pharmaceutically acceptable carrier. Also provided herein are methods of modulating TRPML channels in a subject, the method comprising administering to the subject a therapeutically effective amount of a compound provided herein, or a pharmaceutically acceptable salt thereof.

In certain embodiments, pharmaceutical compositions containing compounds described herein such as a compound of Formula I or Ia, or pharmaceutically acceptable salt thereof can be used to treat or ameliorate a disorder described herein, for example, a ciliopathy.

The amount and concentration of compounds of Formula I or Ia in the pharmaceutical compositions, as well as the quantity of the pharmaceutical composition administered to a subject, can be selected based on clinically relevant factors, such as medically relevant characteristics of the subject (e.g., age, weight, gender, other medical conditions, and the like), the solubility of compounds in the pharmaceutical compositions, the potency and activity of the compounds, and the manner of administration of the pharmaceutical compositions. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

While it is possible for a compound disclosed herein to be administered alone, it is preferable to administer the compound as a pharmaceutical formulation, where the compound is combined with one or more pharmaceutically acceptable diluents, excipients or carriers. The compounds according to the disclosure may be formulated for administration in any convenient way for use in human or veterinary medicine. In certain embodiments, the compound included in the pharmaceutical preparation may be active itself, or may be a prodrug. e.g., capable of being converted to an active compound in a physiological setting. Regardless of the route of administration selected, the compounds of the present disclosure, which may be used in a suitable hydrated form, and/or the pharmaceutical compositions of the present disclosure, are formulated into pharmaceutically acceptable dosage forms such as described below or by other conventional methods known to those of skill in the art.

Thus, another aspect of the present disclosure provides pharmaceutically acceptable compositions comprising a therapeutically effective amount of one or more of the compounds described above, formulated together with one or more pharmaceutically acceptable carriers (additives) and/or diluents. As described in detail below, the pharmaceutical compositions of the present disclosure may be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally: (8) transmucosally; (9) nasally; or (10) intrathecally. Additionally, compounds can be implanted into a patient or injected using a drug delivery system. See, for example, Urquhart, et al., (1994) Ann Rev Pharmacol Toxicol 24:199-236: Lewis, ed. “Controlled Release of Pesticides and Pharmaceuticals” (Plenum Press, New York, 1981); U.S. Pat. No. 3,773,919; and U.S. Patent No. 35 3,270,960.

The phrase “therapeutically effective amount” as used herein means that amount of a compound, material, or composition comprising a compound of the present disclosure, which is effective for producing some desired therapeutic effect, e.g., by modulating EHMT1 or EHMT2, in at least a sub-population of cells in an animal and thereby blocking the biological consequences of that function in the treated cells, at a reasonable benefit/risk ratio applicable to any medical treatment.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent or encapsulating material, involved in carrying or transporting the subject antagonists from one organ, or portion of the body, to another organ, or portion of the body. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as com starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, com oil and soybean oil; (10) glycols, such as propylene glycol: (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) cyclodextrins such as Captisol® and (22) other non-toxic compatible substances employed in pharmaceutical formulations.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66: 1-19 (1977)). Certain specific compounds of the present disclosure contain both basic and acidic functionalities that allow the compounds to be converted into either base or acid addition salts. These salts may be prepared by methods known to those skilled in the art. Other pharmaceutically acceptable carriers known to those of skill in the art are suitable for the present disclosure.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present disclosure include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Formulations of the disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present disclosure as an active ingredient. A compound of the present disclosure may also be administered as a bolus, electuary or paste.

In solid dosage forms of the disclosure for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid: (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate: (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present disclosure, such as dragees, capsules, pills, and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the disclosure include pharmaceutically acceptable emulsions, microemulsions, solutions, suspensions, syrups, and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the disclosure for rectal, vaginal, or urethral administration may be presented as a suppository, which may be prepared by mixing one or more compounds of the disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Alternatively or additionally, compositions can be formulated for delivery via a catheter, stent, wire, or other intraluminal device. Delivery via such devices may be especially useful for delivery to the heart, lung, bladder, urethra, ureter, rectum, or intestine. Furthermore, compositions can be formulated for delivery via a dialysis port.

Ophthalmic formulations, eye ointments, powders, solutions and the like, are also contemplated as being within the scope of this disclosure.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the compositions are administered by intravenous infusion or injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion. Pharmaceutical compositions of this disclosure suitable for parenteral administration comprise one or more compounds of the disclosure in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

When the compounds of the present disclosure are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The addition of the active compound of the disclosure to animal feed is preferably accomplished by preparing an appropriate feed premix containing the active compound in an effective amount and incorporating the premix into the complete ration. Alternatively, an intermediate concentrate or feed supplement containing the active ingredient can be blended into the feed. The way in which such feed premixes and complete rations can be prepared and administered are described in reference books (such as “Applied Animal Nutrition”, W. H. Freedman and CO., San Francisco, U.S.A., 1969 or “Livestock Feeds and Feeding” O and B books, Corvallis. Ore. U.S.A. 1977).

Methods of introduction may also be provided by rechargeable or biodegradable devices. Various slow release polymeric devices have been developed and tested in vivo in recent years for the controlled delivery of drugs, including proteinacious biopharmaceuticals. A variety of biocompatible polymers (including hydrogels), including both biodegradable and non-degradable polymers, can be used to form an implant for the sustained release of a compound at a particular target site.

Preferably, the subject is a mammal. The mammal can be a human, non-human primate, mouse, rat, dog, cat, horse, or cow, but are not limited to these examples. Mammals other than humans can be advantageously used as subjects that represent animal models of disorders associated with neurodegenerative disease or disorder, cancer, or viral infections.

In addition, the methods described herein can be used to treat domesticated animals and/or pets. A subject can be male or female. A subject can be one who has been previously diagnosed with or identified as suffering from or having a neurodegenerative disease or disorder, a disease or disorder associated with cancer, a disease or disorder associated with viral infection, or one or more complications related to such diseases or disorders but need not have already undergone treatment.

### Dosages

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present disclosure employed, or the ester, salt or amide thereof, the route of administration, the time of administration, the rate of excretion of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

The compound and the pharmaceutically active agent can be administrated to the subject in the same pharmaceutical composition or in different pharmaceutical compositions (at the same time or at different times). When administrated at different times, the compound and the pharmaceutically active agent can be administered within 5 minutes, 10 minutes. 20 minutes, 60 minutes, 2 hours, 3 hours, 4, hours, 8 hours, 12 hours, 24 hours of administration of the other agent. When the compound and the pharmaceutically active agent are administered in different pharmaceutical compositions, routes of administration can be different.

The amount of compound that can be combined with a carrier material to produce a single dosage form will generally be that amount of the compound that produces a therapeutic effect. Generally, out of one hundred percent, this amount will range from about 0.1% to 99% of compound, preferably from about 5% to about 70%, most preferably from 10% to about 30%.

Toxicity and therapeutic efficacy can be determined by standard pharmaceutical procedures in cell cultures or experimental animals. e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compositions that exhibit large therapeutic indices are preferred.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized.

The therapeutically effective dose can be estimated initially from cell culture assays. A dose may be formulated in animal models to achieve a circulating plasma concentration range that includes the EC₅₀ (i.e., the concentration of the therapeutic which achieves a half-maximal effect) as determined in cell culture. Levels in plasma may be measured, for example, by high performance liquid chromatography. The effects of any particular dosage can be monitored by a suitable bioassay.

The dosage may be determined by a physician and adjusted, as necessary, to suit observed effects of the treatment.

With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment or make other alteration to treatment regimen. The dosing schedule can vary from once a week to daily depending on a number of clinical factors, such as the subject's sensitivity to the drugs. The desired dose can be administered at one time or divided into subdoses, e.g., 2-4 subdoses and administered over a period of time, e.g., at appropriate intervals through the day or other appropriate schedule. Such sub-doses can be administered as unit dosage forms. In some embodiments, administration is chronic, e.g., one or more doses daily over a period of weeks or months. Examples of dosing schedules are administration daily, twice daily, three times daily or four or more times daily over a period of 1 week, 2 weeks. 3 weeks, 4 weeks, 1 month, 2 months. 3 months, 4 months, 5 months, or 6 months or more.

The present disclosure contemplates formulation of the subject compounds in any of the aforementioned pharmaceutical compositions and preparations. Furthermore, the present disclosure contemplates administration via any of the foregoing routes of administration. One of skill in the art can select the appropriate formulation and route of administration based on the condition being treated and the overall health, age, and size of the patient being treated.

### Selected Chemical Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, propyl, butyl, pentyl, and hexyl.

For compounds of the disclosure in which a variable appears more than once, each variable can be a different moiety selected from the Markush group defining the variable. For example, where a structure is described having two R groups that are simultaneously present on the same compound; the two R groups can represent different moieties selected from the Markush group defined for R.

It is further appreciated that certain features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

In case a compound of the present disclosure is depicted in form of a chemical name and as a formula in case of any discrepancy the formula shall prevail.

An asterisk or wavy line may be used in sub-formulas to indicate the bond which is connected to the core molecule as defined.

The term “substituted,” as used herein, means that any one or more hydrogens on the designated atom, usually a carbon, oxygen, or nitrogen atom, is replaced with a selection from the indicated group, provided that the designated atom's normal valency is not exceeded, and that the substitution results in a stable compound. When a substituent is keto or oxo (i.e., ═O), then 2 hydrogens on the atom are replaced. Ring double bonds, as used herein, are double bonds that are formed between two adjacent ring atoms (e.g., C═C, C═N, N═N, etc.).

As used herein. “alkyl” is intended to include both branched and straight-chain saturated aliphatic hydrocarbon groups having the specified number of carbon atoms. For example, C₁₋₄ alkyl is intended to include C₁, C₂, C₃, and C₄. C₁₋₆ alkyl is intended to include C₁, C₂, C₃, C₄, C₅, and C₆ alkyl groups and C₁₋₈ alkyl is intended to include C₁, C₂, C₃, C₄, C₅, C₆, C₇, and C₈. Some examples of alkyl include, but are not limited to, methyl, ethyl, n-propyl, iso-propyl, n-butyl, s-butyl, t-butyl, n-pentyl, s-pentyl, n-hexyl, n-heptyl, and n-octyl.

As used herein, “alkenyl” is intended to include hydrocarbon chains of either straight or branched configuration and one or more unsaturated carbon-carbon bond that can occur in any stable point along the chain, such as ethenyl and propenyl. For example, C₂₋₆ alkenyl is intended to include C₂, C₃, C₄, C₅, and C₆ alkenyl groups and C₂₋₈ alkenyl is intended to include C₂, C₃, C₄, C₅, C₆, C₇, and C₈ alkenyl groups.

As used herein. “alkylene” is intended to include moieties which are diradicals. i.e., having two points of attachment. A non-limiting example of such an alkylene moiety that is a diradical is —CH₂CH₂—, i.e., a C₂ alkyl group that is covalently bonded via each terminal carbon atom to the remainder of the molecule. The alkylene diradicals are also known as “alkylenyl” radicals. Alkylene groups can be saturated or unsaturated (e.g., containing —CH═CH— or —C≡C—subunits), at one or several positions. In some embodiments, alkylene groups include 1 to 9 carbon atoms (for example, 1 to 6 carbon atoms, 1 to 4 carbon atoms, or 1 to 2 carbon atoms). Some examples of alkylene groups include, but are not limited to, methylene, ethylene, n-propylene, iso-propylene, n-butylene, iso-butylene, sec-butylene, tert-butylene, n-pentylene, iso-pentylene, sec-pentylene and neo-pentylene.

As used herein, “cycloalkyl” is intended to include saturated or unsaturated nonaromatic ring groups, such as cyclopropyl, cyclobutyl, cyclopentyl, or cyclohexyl. For example, the term “C₃₋₈ cycloalkyl” is intended to include C₃, C₄, C₅, C₆, C₇, and C₈ cycloalkyl groups. Cycloalkyls may include multiple spiro- or fused or bridged rings. For example, cycloalkyl can include, but is not limited to, spiro butyl, pentyl, hexyl, heptyl, octyl, nonyl, or decyl groups, bicyclo butyl, pentyl, hexyl, heptyl, octyl, nonyl, or decyl groups, adamantyl groups, and norbornyl groups.

As used herein, the term “heterocycloalkyl” refers to a saturated or unsaturated nonaromatic 3-8 membered monocyclic, 7-12 membered bicyclic (fused, bridged, or spiro rings), or 11-14 membered tricyclic ring system (fused, bridged, or spiro rings) having one or more heteroatoms (such as O, N, S, or Se), unless specified otherwise. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. Examples of heterocycloalkyl groups include, but are not limited to, piperidinyl, piperazinyl, pyrrolidinyl, dioxanyl, tetrahydrofuranyl, isoindolinyl, indolinyl, imidazolidinyl, pyrazolidinyl, oxazolidinyl, isoxazolidinyl, thiazolidinyl, tetrahydrofuranyl, oxiranyl, azetidinyl, oxetanyl, thietanyl, 1,2,3, 6-tetrahydropyridinyl, tetrahydropyranyl, dihydropyranyl, pyranyl, morpholinyl. 1,4-diazepanyl, 1,4-oxazepanyl, 2-oxa-5-azabicyclo[2.2.1]heptanyl, 2,5-diazabicyclo[2.2.1]heptanyl, 2-oxa-6-azaspiro[3.3]heptanyl, 2,6-diazaspiro[3.3]heptanyl, 1,4-dioxa-8-azaspiro[4.5]decanyl and the like.

As used herein. “amine” or “amino” refers to unsubstituted —NH₂ unless otherwise specified. As used herein, “halo” or “halogen” refers to fluoro, chloro, bromo, and iodo substituents.

As used herein, “haloalkyl” is intended to include both branched and straight-chain saturated aliphatic hydrocarbon groups having the specified number of carbon atoms, substituted with one or more halogen (for example —CᵥF_{w}H_{2v.w+1} wherein v=1 to 3 and w=1 to (2v+1)).

Examples of haloalkyl include, but are not limited to, trifluoromethyl, trichlorom ethyl, pentafluoroethyl, and pentachloroethyl.

The term “haloalkoxy” as used herein refers to an alkoxy group, as defined herein, which is substituted one or more halogen. Examples of haloalkoxy groups include, but are not limited to, tnfluoromethoxy, difluoromethoxy, pentafluoroethoxy, trichloromethoxy, etc.

As used herein, “alkoxyl” or “alkoxy” refers to an alkyl group as defined above with the indicated number of carbon atoms attached through an oxygen bridge. C₁₋₆alkoxy, is intended to include C₁, C₂, C₃, C₄, C₅, and C₆ alkoxy groups. C₁₋₈ alkoxy, is intended to include C₁, C₂, C₃, C₄, C₅, C₆, C₇, and C₈ alkoxy groups. Examples of alkoxy include, but are not limited to, methoxy, ethoxy, n-propoxy, i-propoxy, n-butoxy, s-butoxy, t-butoxy, n-pentoxy, s-pentoxy, n-heptoxy, and n-octoxy.

As used herein, “aryl” includes groups with aromaticity, including “conjugated,” or multicyclic systems with at least one aromatic ring and do not contain any heteroatom in the ring structure. Aryl may be monocyclic or polycyclic (e.g., having 2, 3 or 4 fused rings). The term “Cₙ₋ₘ aryl” refers to an aryl group having from n to m ring carbon atoms. In some embodiments, aryl groups have from 6 to 10 carbon atoms. In some embodiments, the aryl group is phenyl or naphthyl.

As used herein, the terms “aromatic heterocycle,” “aromatic heterocyclic” or “heteroaryl” ring are intended to mean a stable 5, 6, 7, 8, 9, 10, 11, or 12-membered monocyclic or bicyclic aromatic ring which consists of carbon atoms and one or more heteroatoms, e.g., 1 or 1-2 or 1-3 or 1-4 or 1-5 or 1-6 heteroatoms, independently selected from nitrogen, oxygen, and sulfur. In the case of bicyclic aromatic heterocyclic or heterocycle or heteroaryl rings, only one of the two rings needs to be aromatic (e.g., 2,3-dihydroindole), though both can be (e.g., quinoline). The second ring can also be fused or bridged as defined above for heterocycles. The nitrogen atom can be substituted or unsubstituted (i.e., N or R wherein R is H or another substituent, as defined). The nitrogen and sulfur heteroatoms can optionally be oxidized (i.e., N→O and S(O)_{P}, wherein p=1 or 2). In certain compounds, the total number of S and O atoms in the aromatic heterocycle is not more than 1.

Examples of aromatic heterocycles, aromatic heterocyclics or heteroaryls include, but are not limited to, acridinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, benzooxadiazolyl, carbazolyl, 4aH-carbazolyl, carbolinyl, cinnolinyl, furazanyl, imidazolyl, imidazolonyl, IH-indazolyl, indolizinyl, indolyl, 3H-indolyl, isobenzofuranyl, isochromanyl, isoindazolyl, isoindolyl, isoquinolinyl, isothiazolyl, isoxazolyl, methylbenztriazolyl, methylfuranyl, methylimidazolyl, methylthiazolyl, naphthyridinyl, oxadiazolyl, 1,2,3-oxadiazolyl, 1,2,4-oxadiazolyl, 1,2,5-oxadiazolyl, 1.3.4-oxadiazolyl, oxazolyl, phenanthridinyl, phenanthrolinyl, phenazinyl, phenothiazinyl, phenoxathinyl, phenoxazinyl, phthalazinyl, pteridinyl, purinyl, pyrazinyl, pyrazolyl, pyridazinyl, pyridooxazolyl, pyridoimidazolyl, pyridothiazolyl, pyridinyl, pyridinonyl, pyridyl, pyrimidinyl, 2H-pyrrolyl, pyrrolyl, quinazolinyl, quinolinyl, 4H-quinolizinyl, quinoxalinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, triazolopyrimidinyl, 1,2,3-triazolyl, 1,2,4-triazolyl, 1,2,5-triazolyl, and 1,3,4-triazolyl.

The term “hydroxyalkyl” means an alkyl group as defined above, where the alkyl group is substituted with one or more OH groups. Examples of hydroxyalkyl groups include HO—CH₂—, HO—CH₂-CH₂— and CH₃-CH(OH)—.

The term “cyano” as used herein means a substituent having a carbon atom joined to a nitrogen atom by a triple bond, i.e., C≡N.

As used herein, “oxo” is means a “═O” group.

As used herein, the phrase “pharmaceutically acceptable” refers to those compounds or tautomers thereof, or salts thereof, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. As used herein. “pharmaceutically acceptable salts” refer to derivatives of the disclosed compounds or tautomers thereof, wherein the parent compound or a tautomer thereof, is modified by making of the acid or base salts thereof of the parent compound or a tautomer thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound, or a tautomer thereof, formed, for example, from non-toxic inorganic or organic acids. For example, such conventional non-toxic salts include, but are not limited to, those derived from inorganic and organic acids selected from 2-acetoxybenzoic, 2-hydroxy ethane sulfonic, acetic, ascorbic, benzene sulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, and toluene sulfonic.

The pharmaceutically acceptable salts of the present disclosure can be synthesized from the parent compound or a tautomer thereof that contains a basic or acidic moiety by conventional chemical methods. Generally, such pharmaceutically acceptable salts can be prepared by reacting the free acid or base forms of these compounds or tautomers thereof with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 18th ed., Mack Publishing Company, Easton, PA, USA, p. 1445 (1990).

As used herein, “stable compound” and “stable structure” are meant to indicate a compound that is sufficiently robust to survive isolation to a useful degree of purity from a reaction mixture, and formulation into an efficacious therapeutic agent.

As used herein, the term “treating” refers to administering a compound or pharmaceutical composition as provided herein for therapeutic purposes. The term “therapeutic treatment” refers to administering treatment to a patient already suffering from a disease thus causing a therapeutically beneficial effect, such as ameliorating existing symptoms, ameliorating the underlying metabolic causes of symptoms, postponing or preventing the further development of a disorder, and/or reducing the severity of symptoms that will or are expected to develop.

As used herein. “unsaturated” refers to compounds having at least one degree of unsaturation (e.g., at least one multiple bond) and includes partially and fully unsaturated compounds.

As used herein, the term “effective amount” refers to an amount of a compound or a pharmaceutically acceptable salt of the compound or tautomer (including combinations of compounds and/or tautomers thereof, and/or pharmaceutically acceptable salts of said compound or tautomer) of the present disclosure that is effective when administered alone or in combination as an antimicrobial agent. For example, an effective amount refers to an amount of the compound or tautomer thereof, or a pharmaceutically acceptable salt said compound or tautomer that is present in a composition, a formulation given to a recipient patient or subject sufficient to elicit biological activity.

In the specification, the singular forms also include the plural, unless the context clearly dictates otherwise. Unless defined otherwise, all technical and scientific terms used herein have the same meaning as commonly understood by one of ordinary skill in the art to which this disclosure belongs. In the case of conflict, the present specification will control. As used herein, “mammal” refers to human and non-human patients.

As used herein, the term “formulae of the disclosure” or “formulae disclosed herein” includes one or more of the Formulas I or Ia and subformulas thereof.

As used herein, the term “compound of the disclosure” or “compound disclosed herein” includes one or more compounds of the formulae of the disclosure or a compound explicitly disclosed herein.

All percentages and ratios used herein, unless otherwise indicated, are by weight.

Throughout the description, where compositions are described as having, including, or comprising specific components, or where processes are described as having, including, or comprising specific process steps, it is contemplated that compositions of the present disclosure also consist essentially of, or consist of, the recited components, and that the processes of the present disclosure also consist essentially of, or consist of, the recited processing steps. Further, it should be understood that the order of steps or order for performing certain actions are immaterial so long as the disclosure remains operable. Moreover, two or more steps or actions can be conducted simultaneously.

Contemplated equivalents of the compounds described above include compounds which otherwise correspond thereto, and which have the same general properties thereof (e.g., the ability to modulate TRPML), wherein one or more simple variations of substituents are made which do not adversely affect the efficacy of the compound. In general, the compounds of the present disclosure may be prepared by the methods illustrated in the general reaction schemes as, for example, described below, or by modifications thereof, using readily available starting materials, reagents and conventional synthesis procedures. In these reactions, it is also possible to make use of variants which are in themselves known, but are not mentioned here.

As used herein, the articles “a” and “an” refer to one or to more than one (e.g., to at least one) of the grammatical object of the article.

“About” and “approximately” shall generally mean an acceptable degree of error for the quantity measured given the nature or precision of the measurements. Exemplary degrees of error are within 20 percent (%), typically, within 10%, and more typically, within 5% of a given value or range of values.

The term, “treat” or “treatment,” as used herein, refers to the application or administration of a compound, alone or in combination with, an additional agent to a subject, e.g., a subject who has a disorder (e.g., a disorder as described herein), a symptom of a disorder, or a predisposition toward a disorder, with the purpose to cure, heal, alleviate, relieve, alter, remedy, ameliorate, improve or affect the disorder.

As used herein, the term “subject” is intended to include human and non-human animals. Exemplary human subjects include a human subject having a disorder, e.g., a disorder described herein. The term “non-human animals” of the disclosure includes all vertebrates, e.g., non-mammals (such as chickens, amphibians, reptiles) and mammals, such as non-human primates, domesticated and/or agriculturally useful animals, e.g., sheep, dog, cat, cow, pig, etc.

The terms “antagonist” and “inhibitor” are used interchangeably to refer to an agent that decreases or suppresses a biological activity.

The terms “activator” and “agonist” are used interchangeably to refer to an agent that increases or initiates a biological activity.

The term “hydrate” as used herein, refers to a compound formed by the union of water with the parent compound.

The term “preventing,” when used in relation to a condition, such as a local recurrence (e.g., pain), a disease such as cancer, a syndrome complex such as heart failure or any other medical condition, is well understood in the art, and includes administration of a composition which reduces the frequency of, or delays the onset of, symptoms of a medical condition in a subject relative to a subject which does not receive the composition. Thus, prevention of cancer includes, for example, reducing the number of detectable cancerous growths in a population of patients receiving a prophylactic treatment relative to an untreated control population, and/or delaying the appearance of detectable cancerous growths in a treated population versus an untreated control population, e.g., by a statistically and/or clinically significant amount. Prevention of an infection includes, for example, reducing the number of diagnoses of the infection in a treated population versus an untreated control population, and/or delaying the onset of symptoms of the infection in a treated population versus an untreated control population. Prevention of pain includes, for example, reducing the magnitude of, or alternatively delaying, pain sensations experienced by subjects in a treated population versus an untreated control population.

The term “solvate” as used herein, refers to a compound formed by solvation (e.g., a compound formed by the combination of solvent molecules with molecules or ions of the solute). Another aspect of the disclosure features a pharmaceutical preparation suitable for use in a human patient, or for veterinary use, comprising an effective amount of a compound of the formulae of the disclosure (or a salt thereof, or a solvate, hydrate, oxidative metabolite or prodrug of the compound or its salt), and one or more pharmaceutically acceptable excipients. The disclosure further contemplates the use of compounds of the formulae of the disclosure in the manufacture of a medicament or pharmaceutical preparation to treat or reduce the symptoms of any of the diseases or conditions provided in the specification. The compounds of the formulae of the disclosure for use in treating a particular disease or condition can be formulated for administration via a route appropriate for the particular disease or condition.

Compounds of the formulae of the disclosure can be administered alone or in combination with another therapeutic agent. For instance, the compounds of the formulae of the disclosure can be administered conjointly with one or more of an agent for treating polycystic kidney disease, etc.

Compounds of the formulae of the disclosure can be administered topically, orally, transdermally, rectally, vaginally, parentally, intranasally, intrapulmonary, intraocularly, intravenously, intramuscularly, intraarterially, intrathecally, intracapsularly, intraorbitally, intracardiacly, intradermally, intraperitoneally, transtracheally, subcutaneously, subcuticularly, intraarticularly, subcapsularly, subarachnoidly, intraspinally, intrastemally, sublingually, or by inhalation.

In some embodiments, compounds of Formula I or Ia can be administered topically.

In some embodiments, compounds of Formula I or Ia can be administered orally.

In some embodiments, compounds of Formula I or Ia can be administered parentally.

Compounds of Formula I or Ia include molecules having an aqueous solubility suitable for oral or parenteral (e.g., intravenous) administration leading to or resulting in the treatment of a disorder described herein, for example the treatment of pain. In some embodiments, the compound is formulated into a composition suitable for oral administration.

In some embodiments, a compound of Formula I or Ia can be administered as part of an oral or parenteral (e.g., intravenous) pharmaceutical composition to treat a disorder described herein in a therapeutically effective manner.

Certain compounds disclosed herein may exist in particular geometric or stereoisomeric forms. The present disclosure contemplates all such compounds, including cis- and trans-isomers, R- and S-enantiomers, diastereomers, (d)-isomers, (l)-isomers, the racemic mixtures thereof, and other mixtures thereof, as falling within the scope of the disclosure. For example, if one chiral center is present in a molecule, the disclosure includes racemic mixtures, enantiomerically enriched mixtures, and substantially enantiomerically or diastereomerically pure compounds. The composition can contain, e.g., more than 50%, more than 60%, more than 70%, more than 80%, more than 90%, more than 95%, or more than 99% of a single enantiomer or diastereomer. Additional asymmetric carbon atoms may be present in a substituent such as an alkyl group. All such isomers, as well as mixtures thereof, are intended to be included in this disclosure.

The “enantiomeric excess” or “% enantiomeric excess” of a composition can be calculated using the equation shown below. In the example shown below a composition contains 90% of one enantiomer, e.g., the S enantiomer, and 10% of the other enantiomer, i.e., the R enantiomer.

$$ee = ( 90 - 10 ) / 100 = 80 \% .$$

Thus, a composition containing 90% of one enantiomer and 10% of the other enantiomer is said to have an enantiomeric excess of 80%.

The “diastereomeric excess” or “% diastereomeric excess” of a composition can be calculated using the equation shown below. In the example shown below a composition contains 90% of one diastereomer, and 10% of another enantiomer.

$$de = ( 90 - 10 ) / 100 = 80 \% .$$

Thus, a composition containing 90% of one diastereomer and 10% of the other diastereomer is said to have a diastereomeric excess of 80%.

Certain compounds disclosed herein can exist in unsolvated forms as well as solvated forms, including hydrated forms. In general, the solvated forms are equivalent to unsolvated forms and are encompassed within the scope of the present disclosure. Certain compounds disclosed herein may exist in multiple crystalline or amorphous forms. In general, all physical forms are equivalent for the uses contemplated by the present disclosure and are intended to be within the scope of the present disclosure.

### EXAMPLES

Examples are provided below to facilitate a more complete understanding of the invention. The following examples illustrate exemplary modes of making and practicing the invention. However, the scope of the invention is not limited to specific embodiments disclosed in these Examples, which are for purposes of illustration only, since alternative methods can be utilized to obtain similar results.

General. All oxygen and/or moisture sensitive reactions were carried out under N₂ atmosphere in glassware that was flame-dried under vacuum (0.5 mmHg) and purged with N₂ prior to use. All reagents and solvents were purchased from commercial vendors and used as received, or synthesized according to the footnoted references. NMR spectra were recorded on a Bruker 400 (400 MHz ¹H, 75 MHz ¹³C) or Varian (400 MHz ¹H, 75 MHz ¹³C) spectrometer. Proton and carbon chemical shifts are reported in ppm (δ) referenced to the NMR solvent. Data are reported as follows: chemical shifts, multiplicity (br=broad, s=singlet, t=triplet, q=quartet, m=multiplet; coupling constant (s) in Hz). Unless otherwise indicated NMR data were collected at 25° C. Flash chromatography was performed using 100-200 mesh Silica Gel. Liquid Chromatography/Mass Spectrometry (LCMS) was performed on Agilent 1200HPLC and 6110MS. Analytical thin layer chromatography (TLC) was performed on 0.2 mm silica gel plates. Visualization was accomplished with UV light and aqueous potassium permanganate (KMn04) stain followed by heating.

### LCMS Tvpical Conditions:

LC-MS Instrument: LCMS2020(E-LCMS 008); Column: Shim-pack GIST C18, 50*4.6 mm 5 m Mobile Phase: Solvent A: H₂O (0.1% FA), Solvent B: CH₃CN; Temperature: 35° C.; Flow rate: 2.5 mL/min; Run time: 0.1 min@20%1B, 1.7 min gradient(20-95% B), then 0.7 min@,95% B, then 0.4 min(@20% B; Injection volume: 5 L; Detector: UV 220/254 nm; Mass range: 100-1000; Scan: Positive/Negative.

### HPLC Typical Conditions:

Instrument: LC-20AD(E-LC 006); Column: YMC Triart C18, 50×4.6 mm,5 μm; Mobile phase: Solvent A: H₂O/CH₃CN/TFA=90/10/0.1, Solvent B: H₂O/CH₃CN/TFA=10/90/0.1; Flow rate: 2.5 mL/min: Run time: 0.4 min(@60% B. 3.4 min gradient (60-100% B), then 0.8 min@k100% B: Temperature: 35° C. Detector: UV.
Or

Instrument: LC-2010AHT(E-LC 001); Column: Gemini, C18, 50×4.6 mm,5 μm; Mobile phase: Solvent A: H₂O/CH₃CN/TFA=90/10/0.1, Solvent B: H₂O/CH₃CN/TFA=10/90/0.1; Flow rate: 2.5 mL/min; Run time: 0.4 min(@) 20% B, 3.4 min gradient (20-95% B), then 0.8 min@0.95% B; Temperature: 40° C.; Detector: UV.

TABLE 2
Abbreviations

| | | | |
|---|---|---|---|
| Boc | t-butoxycarbonyl | LCMS | liquid chromatography mass |
| DCM | dichloromethane | | spectrometry |
| DIEA or | N,N-diisopropylethylamine | HPLC | High performance liquid |
| DIPEA | | | chromatography |
| dppf | 1,1′- | Me | methyl |
| | bis(diphenylphosphino)ferrocene | MeOH | methanol |
| ESI | electrospray ionization | Pd(dppf)Cl₂ | 1,1- |
| EtOAc | ethyl acetate | | bis(diphenylphosphino)ferrocene- |
| EtOH | ethanol | | palladium(II)dichloride |
| HATU | O-(7-azabenzotriazol-1-yl)- | TBAF | tetrabutylammonium fluoride |
| | N,N,N′,N′- | TEA | triethylamine |
| | tetramethyluronium | TFA | trifluoroacetic acid |
| | hexafluorophosphate | THF | tetrahydrofuran |
| ¹H NMR | proton nuclear magnetic | TLC | thin layer chromatography |
| | resonance | Ts | p-toluenesulfonyl |
| | | TsCl | 4-toluenesulfonyl chloride |

### General Synthesis of Boc-Protected Intermediates

The syntheses of Boc-protected intermediates described herein are illustrated by the following representative example. Analogous Boc-protected intermediates can be obtained following suitable procedures with appropriate reagents (e.g., aryl halides, boronic acids/esters, piperazines, etc.).

### Example 1. Synthesis of tert-butyl (2R,5S)-4-(7-(3-cyanophenyl)-5-phenyl-7H-pyrrolo[2,3-d]pyrimidin-4-yl)-2,5-dimethylpiperazine-1-carboxylate

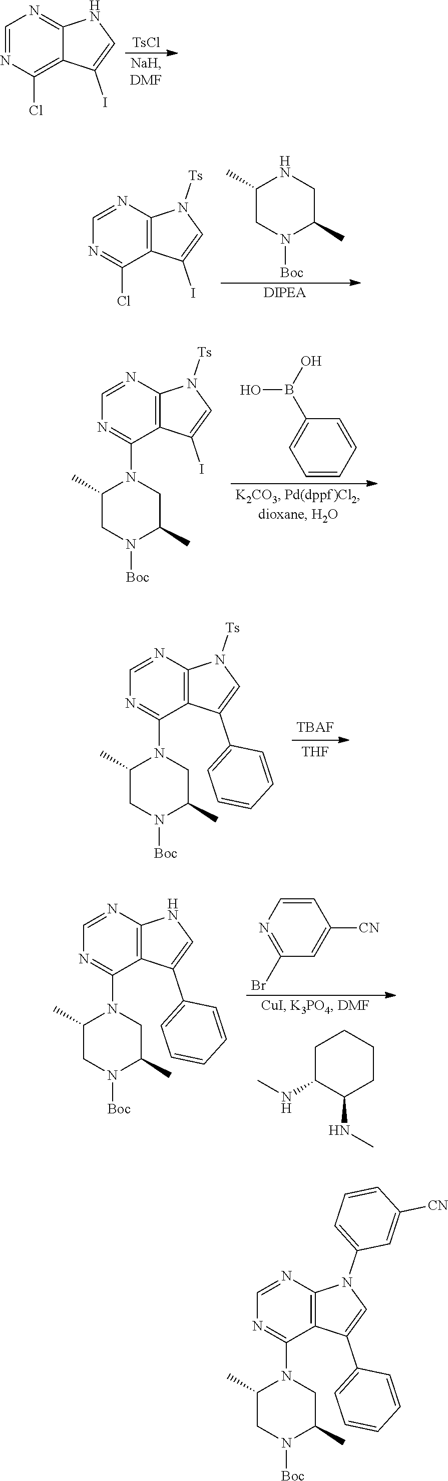

### Step 1. 4-Chloro-5-iodo-7-tosyl-7H-pyrrolo[2,3-d]pyrimidine

To a suspension of NaH (1.0 g, 27 mmol, 60% wt %) in anhydrous DMF (60 mL) at 0° C. was added 4-chloro-5-iodo-7H-pyrrolo[2,3-d]pyrimidine (5.0 g, 18 mmol) in portions. The resulting mixture was stirred at 0° C. for 30 minutes before TosCl (3.4 g. 18 mmol) was added in portions. After the addition, the reaction was stirred at room temperature overnight. The reaction was poured into ice water, filtered. The solid was collected and further dried in vacuo to give 4-chloro-5-iodo-7-tosyl-7H-pyrrolo[2,3-d]pyrimidine (6.0 g, 77%) as a white solid. LCMS ESI m/z: 434 (M+H)⁻.

### Step 2. tert-Butyl (2R,5S)-4-(5-iodo-7-tosyl-7H-pyrrolo[2.3-dlpyrimidin-4-yl)-2,5-dimethylpiperazine-1-carboxylate

To a solution of 4-chloro-5-iodo-7-tosyl-7H-pyrrolo[2,3-d]pyrimidine (4.0 g, 9.2 mmol) in DIPEA (5.0 mL, 28 mmol) was added tert-butyl (2R,5S)-2.5-dimethylpiperazine-1-carboxylate (2.0 g, 9.2 mmol). The resulting mixture was heated to 150° C. for 3 h under a N₂ atmosphere. After cooling to room temperature, solvent was removed and the residue was purified by flash column chromatography (silica gel, 0~30%, ethyl acetate in petroleum ether) to afford tert-butyl (2R,5S)-4-(5-iodo-7-tosyl-7H-pyrrolo[2,3-d]pyrimidin-4-yl)-2,5-dimethylpiperazine-1-carboxylate (2.5 g, 43%) as a yellow solid. LC/MS ESI m/z: 612 (M+H)⁺.

### Step 3. tert-Butyl (2R,5S)-2,5-dimethyl-4-(5-phenyl-7-tosyl-7H-pyrrolo[2,3-d]pyrimidin-4-yl)piperazine-]-carboxylate

To a solution of tert-butyl (2R,5S)-4-(5-iodo-7-tosyl-7H-pyrrolo[2,3-d]pyrimidin-4-yl)-2,5-dimethylpiperazine-1-carboxylate (150 mg, 0.30 mmol) in dioxane (10 mL) and H₂O (1 mL) were added phenylboronic acid (60 mg, 0.60 mmol), K₂CO₃ (100 mg, 0.75 mmol) and Pd(dppf)C₁₂ (18 mg, 0.010 mmol). The resulting mixture was stirred at 90° C. overnight under a N₂ atmosphere. After cooling to room temperature, solvent was removed and the residue was purified by flash column chromatography (silica gel, 0~20%, ethyl acetate in petroleum ether) to afford tert-butyl (2R,5S)-2,5-dimethyl-4-(5-phenyl-7-tosyl-7H-pyrrolo[2,3-d]pyrimidin-4-yl)piperazine-1-carboxylate (100 mg, 72%) as a yellow solid. LC/MS ESI m/z: 562 (M+H)⁺.

### Step 4. tert-Butyl (2R.5S)-2,5-dimethyl-4-(5-phenyl-7H-pyrrolo[2.3-d]pyrimidin-4-yl)piperazine-1-carboxylate

To a solution of tert-butyl (2R,5S)-2,5-dimethyl-4-(5-phenyl-7-tosyl-7H-pyrrolo[2,3-d]pyrimidin-4-yl)piperazine-1-carboxylate (100 mg, 0.18 mmol) in THF (2 mL) was added TBAF (5 mL). The resulting mixture was stirred at room temperature overnight. The reaction was quenched with water and extracted twice with EtOAc. The combined organic layers were washed with brine, dried over Na₂SO₄, filtered and concentrated. The residue was purified by flash column chromatography (silica gel, 0~60%, ethyl acetate in petroleum ether) to afford tert-butyl (2R,5S)-2,5-dimethyl-4-(5-phenyl-7H-pyrrolo[2,3-d]pyrimidin-4-yl)piperazine-1-carboxylate (50 mg, 69%) as a white solid. LC/MS ESI m/z: 408 (M+H)⁺.

### Step 5. tert-Butyl (2R,5S)-4-(7-(3-cyanophenyl)-5-phenyl-7H-pyrrolo[2,3-d]pyrimidin-4-yl)-2,5-dimethylpiperazine-1-carboxylate

To a solution of tert-butyl (2R,5S)-2,5-dimethyl-4-(5-phenyl-7H-pyrrolo[2,3-d]pyrimidin-4-yl)piperazine-1-carboxylate (4.0 g. 9.8 mmol) in DMF (80 mL) were added 3-bromobenzonitrile (3.6 g, 20 mmol), CuI (1.9 g, 9.8 mmol), trans-cyclohexane-1,2-diamine (2.8 g, 20 mmol) and K₃PO₄ (4.2 g, 20 mmol). The resulting mixture was heated to 100° C. overnight.

After cooling to room temperature, the reaction was partitioned between EtOAc and water. The aqueous layer was extracted twice with EtOAc. The combined organic layers were washed with brine, dried over Na₂SO₄, filtered and concentrated. The residue was purified by flash column chromatography (silica gel, 0-40%, ethyl acetate in petroleum ether) to afford tert-butyl (2R,5S)-4-(7-(3-cyanophenyl)-5-phenyl-7H-pyrrolo[2.3-d]pyrimidin-4-vl)-2,5-dimethylpiperazine-1-carboxylate (4.6 g, 92%) as a yellow solid. LC/MS ESI m/z: 509 (M+H)*.

### Example 2. Synthesis of 3-(4-((2S,5R)-4-(2-hydroxy-2-methylpropanoyl)-2,5-dimethylpiperazin-1-yl)-5-phenyl-7H-pyrrolo[2,3-d]pyrimidin-7-yl)benzonitrile (Compound 101)

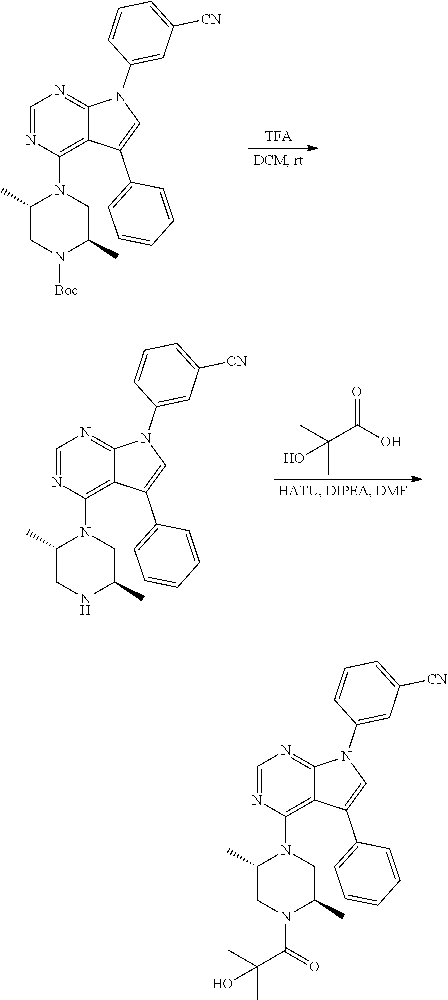

### Step 1. 3-(4-((2S,5R)-2,5-Dimethylpiperazin-1-yl)-5-phenyl-7H-pyrrolo[2,3-d]pyrimidin-7-yl)benzonitrile

To a solution of tert-butyl (2R,5S)-4-(7-(3-cyanophenyl)-5-phenyl-7H-pyrrolo[2,3-d]pyrimidin-4-yl)-2,5-dimethylpiperazine-1-carboxylate (4.6 g, 8.9 mmol) in DCM (20 mL) was added TFA (15 mL) at 0° C. After stirring at room temperature for 1 h, the reaction was concentrated and the residue (3.7 g, yellow oil) was used directly in the next step. LC/MS ESI m/z: 409 (M+H)⁺.

### Step 2. 3-(4-((2S,5R)-4-(2-Hydroxy-2-methylpropanoyl)-2,5-dimethylpiperazin-1-yl)-5-phenyl-7H-pyrrolo[2,3-d]pyrimidin-7-yl)benzonitrile

To a solution of 3-(4-((2S,5R)-2,5-dimethylpiperazin-1-yl)-5-phenyl-7H-pyrrolo[2,3-d]pyrimidin-7-yl)benzonitrile (3.7 g, 8.9 mmol) in DMF (100 mL) were added 2-hydroxy-2-methylpropanoic acid (1.9 g. 18 mmol), HATU (6.8 g, 18 mmol) and DIPEA (7.4 mL. 45 mmol). After stirring at room temperature overnight, the reaction was partitioned between EtOAc and NaHCO₃(aq.). The aqueous layer was extracted twice with EtOAc. The combined organic layers were washed with brine, dried over Na₂SO₄, filtered and concentrated. The residue was purified by flash column chromatography (silica gel. 0~50%, ethyl acetate in petroleum ether) to give crude product which was further purified by reverse phase Cis column (30%~100%, MeCN in water) to afford 3-(4-((2S,5R)-4-(2-hydroxy-2-methylpropanoyl)-2,5-dimethylpiperazin-1-yl)-5-phenyl-7H-pyrrolo[2,3-d]pyrimidin-7-yl)benzonitrile (1.2 g, 28%) as a white solid. LC/MS ESI m/z: 495 (M+H)⁻. ¹H NMR (400 MHz, CDCl₃) δ 8.54 (s. 1H), 8.17 (d. J=1.0 Hz. 1H), 8.08 (m. 1H), 7.66 (m, 2H), 7.55 (d, J=7.1 Hz, 2H), 7.49 (t, J=7.5 Hz, 2H), 7.43-7.35 (m, 2H), 4.65 (br. s, 0.5H), 4.55-4.15 (m, 2H), 3.97 (br. s, 0.5H), 3.83 (br. s, 0.5H), 3.57 (br. s, 0.5H), 3.45 (m, 1.5H), 3.28 (br. s, 1H). 2.71 (br. s, 0.5H), 1.55-1.25 (m, 7.5H), 1.07 (m. 3H), 0.78 (br. s, 1.5H); several peaks and their integrations are fractions of protons on account of presence of rotamers.

The following compounds were prepared by procedures analogous to the synthesis of Compound 101 using the corresponding Boc-protected piperazine and carboxylic acid. In the ¹H NMR spectra of some analogs, several peaks and their integrations are fractions of protons on account of presence of rotamers.

| Ex | | | |
|---|---|---|---|
| No. | Structure | Name | LCMS and ¹H NMR |
| 102 | 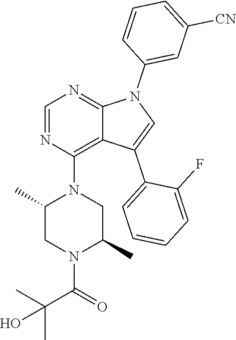 [SMILES: C[C@@H]1CN(c2ncnc3c2c(-c2ccccc2F)cn3-c2cccc(C#N)c2)[C@@H](C)CN1C(=O)C(C)(C)O] | 3-(5-(2- fluorophenyl)-4- ((25,5R)-4-(2- hydroxy-2- methylpropanoyl)- 2,5- dimethylpiperazin- 1-yl)-7H- pyrrolo[2,3- d]pyrimidin-7- yl)benzonitrile | LC/MS ESI m/z: 513 (M + H)⁺. ¹H NMR (400 MHz, CDCl₃) δ 8.53 (s, 1H), 8.17 (s, 1H), 8.07 (m, 1H), 7.66 (m, 2H), 7.53-7.45 (m, 2H), 7.40 (m, 1H), 7.30-7.20 (m, 2H), 4.69- 4.18 (m, 2.5H), 4.06-3.84 (m, 1H), 3.63- 3.20 (m, 3H), 2.66 (br. s, 0.5H), 1.53-1.35 (m, 7.5H), 1.08 (br. s, 3H), 0.82 (br. s, 1.5H). |
| 103 | 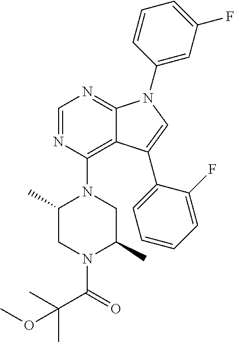 [SMILES: COC(C)(C)C(=O)N1C[C@H](C)N(c2ncnc3c2c(-c2ccccc2F)cn3-c2cccc(F)c2)C[C@H]1C] | 1-((2R,5S)-4-(5- (2-fluorophenyl)- 7-(3- fluorophenyl)-7H- pyrrolo[2,3- d]pyrimidin-4-yl)- 2,5- dimethylpiperazin- 1-yl)-2-methoxy- 2-methylpropan- 1-one | LC/MS ESI m/z: 520 (M + H)⁺. ¹H NMR (400 MHz, CDCl₃) δ 8.55 (s, 1H), 7.61-7.44 (m, 5H), 7.37 (m, 1H), 7.30-7.17 (m, 2H), 7.10 (m, 1H), 5.15 (br. s, 0.5H), 4.63 (br. s, 0.5H), 4.23 (br. s, 0.5H), 4.06 (m, 1H), 3.88 (d, J = 13.7 Hz, 0.5H), 3.57-3.33 (m, 2H), 3.30- 3.25 (m, 2H), 3.14 (s, 1.5H), 2.63 (m, 0.5H), 1.45 (s, 1.5H), 1.43 (s, 1.5H), 1.40 (s, 1.5H), 1.34 (s, 3H), 1.08 (d, J = 6.6 Hz, 3H), 0.84 (d, J = 5.3 Hz, 1.5H). |
| 104 | 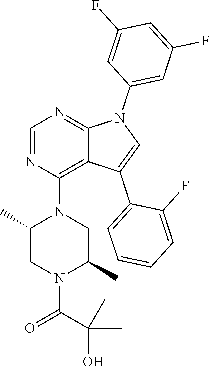 [SMILES: C[C@@H]1CN(c2ncnc3c2c(-c2ccccc2F)cn3-c2cc(F)cc(F)c2)[C@@H](C)CN1C(=O)C(C)(C)O] | 1-((2R,5S)-4-(7- (3,5- difluorophenyl)-5- (2-fluorophenyl)- 7H-pyrrolo[2,3- d]pyrimidin-4-yl)- 2,5- dimethylpiperazin- 1-yl)-2-hydroxy- 2-methylpropan- 1-one | LC/MS (ESI) m/z: 524 (M + H)⁺. ¹H NMR (400 MHz, CDCl₃) δ 8.54 (s, 1H), 7.52-7.37 (m, 5H), 7.31-7.19 (m, 2H), 6.83 (m, 1H), 4.66-4.23 (m, 2.5H), 4.04-3.81 (m, 1H), 3.61-3.13 (m, 3II), 2.64 (m, 0.5II), 1.53- 1.34 (m, 7.5H), 1.19-0.96 (m, 3H), 0.87- 0.74 (m, 1.5H). |
| 105 | 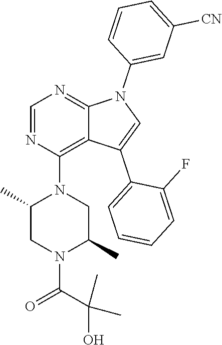 | 1-((2R,5S)-4-(7- (3-chlorophenyl)- 5-(2- fluorophenyl)-7H- pyrrolo[2,3- d]pyrimidin-4-yl)- 2,5- dimethylpiperazin- 1-yl)-2-hydroxy- 2-methylpropan- 1-one | LC/MS ESI m/z: 522 (M + H)⁺. ¹H NMR (400 MHz, CDCl₃) δ 8.53 (s, 1H), 7.80 (s, 1H), 7.69 (m, 1H), 7.55-7.43 (m, 3H), 7.37 (m, 2H), 7.30-7.18 (m, 2H), 4.70-4.15 (m, 2.5H), 4.08-3.76 (m, 1H), 3.74-3.04 (m, 3H), 2.64 (m, 0.5H), 1.54-1.36 (m, 7.5H), 1.07 (br. s, 3H), 0.81 (br. s, 1.5H). |

### Example 3. Fluorescent TRPML Assays

### TRPML1 Assay

### Cell Culture

HEK-293 Trex cells were stably transfected with a construct consisting of the human coding sequence for TRPML1 cloned into the tet-inducible plasmid pCDNA5 T/O. Mutations were introduced into the TRPML1 sequence to facilitate expression on the cell surface (Silvia Vergarajauregui. Rosa Puertollano Traffic. 2006 March: 7(3): 337-353). Briefly, the cells are cultured in 150 mm round tissue culture dishes containing 20 mL of media. The day before the assay the cells are rinsed with DPBS—Ca —Mg and then treated briefly with Trypsin-EDTA. The Trypsin-EDTA is diluted with growth media, and cells are counted. 38×10\hat{ }6 cells are re-plated into 150 mm round tissue culture dishes in media containing 0.5 g/mL doxycycline to induce expression of hTRPML1.

### Dye Loading

The day of the experiment cells are lifted from the plates as above and collected by centrifugation. The cells are then suspended in dye loading buffer consisting of Ringer's solution supplemented with 0.1% Pluronic Acid and 1 micromolar Fluo4-AM dye. Cells are loaded for ~60 minutes in the dark with occasional mixing. The cells are collected by centrifugation, the loading media aspirated, and the cells resuspended in 25 mL Ringer's solution and incubated ~60 minutes in the dark. The cells are again collected by centrifugation, rinsed in Ringer's Solution and resuspended to 0.2×10\hat{ }6 cells/mL in modified Ringer's solution containing 10 mM calcium.

### Compound Assay Plates

Compounds are dissolved to a concentration of 10 millimolar with DMSO. Compound plates are created by dispensing compounds into 384 well black wall clear bottom plates (Greiner 781091). Positive and negative controls are included on each plate. Typically, different amounts of each compound are tested ranging from 100 nanoMoles (20 micromolar final concentration) decreasing in half-log steps to 31 picoMoles (6 nanomolar final concentration). Each concentration is typically tested in triplicate.

### Assay

50 microliters of dye-loaded cells are dispensed into each well of the compound assay plate created above. The fluorescence in each well is then determined with an excitation wavelength of 480 nM and an emission wavelength of 540 nM using either a Molecular Devices SpectraMax multimode plate reader or a Hamamatsu FDSS/uCell plate imager.

### Analysis and Statistics

The resulting fluorescence for each well is exported as an ascii file and loaded into our LIMS for analysis. The percent activity of each compound at each concentration is determined by comparison to the positive and negative control wells included in each plate.

### TRPML2 and TRPML3 Assays

Assays for TRPML2 and TRPML3 were performed as above for TRPML1, by substituting the appropriate TRPML2 or TRPML3 subtype for the TRPML1.

EC₅₀ values were calculated using a non-linear regression of Prism. The EC₅₀ determined for each compound using the assay is summarized in Table 3 below. The compound numbers correspond to those shown in Table 1. In the table, “A” indicates an EC₅₀ of less than 100 nM, “B” indicates an EC₅₀ range from 100 nM to 500 nM; “C” indicates an EC₅₀ range from 500 nM to 2 μM; and “D” indicates an EC₅₀ greater than 2 μM.

TABLE 3
Efficacy of exemplary compounds of the invention

| | Human | Human | Human |
|---|---|---|---|
| Cmpd | TRPML1:EC₅₀ | TRPML2:EC₅₀ | TRPML3:EC₅₀ |
| No. | (nM) | (nM) | (nM) |
| 101 | B | — | D |
| 102 | B | — | D |
| 103 | A | B | A |
| 104 | C | — | D |
| 105 | B | — | C |

### EQUIVALENTS

It will be recognized that one or more features of any embodiments disclosed herein may be combined and/or rearranged within the scope of the invention to produce further embodiments that are also within the scope of the invention.

Those skilled in the art will recognize or be able to ascertain using no more than routine experimentation, many equivalents to the specific embodiments of the invention described herein. Such equivalents are intended to be within the scope of the present invention.

Although the invention has been described and illustrated in the foregoing illustrative embodiments, it is understood that the present disclosure has been made only by way of example, and that numerous changes in the details of implementation of the invention can be made without departing from the spirit and scope of the invention, which is limited only by the claims that follow. Features of the disclosed embodiments can be combined and/or rearranged in various ways within the scope and spirit of the invention to produce further embodiments that are also within the scope of the invention. Those skilled in the art will recognize, or be able to ascertain, using no more than routine experimentation, numerous equivalents to the specific embodiments described specifically in this disclosure. Such equivalents are intended to be encompassed in the scope of the following claims.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

1. A compound of formula (I) 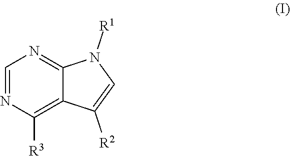
or a pharmaceutically acceptable salt thereof, wherein
R¹ is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b};
R² is aryl optionally substituted with one or more substituents each independently selected from the group consisting of halogen, hydroxy, cyano, C₁₋₆ alkyl, C₁₋₃ haloalkyl, C₁₋₆ alkoxy, C₁₋₆ haloalkoxy, C₁₋₆ alkylthio, C₃₋₇ cycloalkyl, and NR^{a}R^{b};
R³ is 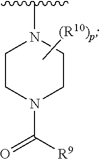
each R¹⁰ is independently selected from the group consisting of C₁₋₆ alkyl, C₁₋₆ haloalkyl, and cycloalkyl, wherein the C₁₋₆ alkyl, C₁₋₆ haloalkyl, and cycloalkyl are optionally substituted with 1-5 substituents independently selected from the group consisting of deuterium, hydroxy, and C₁₋₃ alkoxy; or
any two R¹⁰ can be taken together with the atoms to which they are attached to form a fused ring of 3-7 members, or a bridge of 1 to 3 carbons or a single bond, wherein the ring or bridge is optionally substituted with 1-3 substituents independently selected from the group consisting of deuterium, halogen, hydroxy, C₁₋₆ haloalkyl, and C₁₋₆ alkyl;
R⁹ is C₁₋₆ alkyl substituted with one or more hydroxy, C₁₋₆ alkoxy or C₁₋₆ haloalkoxy and optionally substituted with one or more substituents each independently selected from deuterium, halogen, C₁₋₃ haloalkyl, and C₁₋₆ haloalkoxy;
each R^{a} and R^{b} is independently selected from H and C₁₋₆ alkyl; and
p is 0, 1, 2, 3, or 4.

2. The compound of claim 1 or a pharmaceutically acceptable salt thereof, wherein R¹ is phenyl optionally substituted with one or more substituents each independently selected from the group consisting of halogen, hydroxy, cyano, C₁₋₆ alkyl, C₁₋₃ haloalkyl, C₁₋₆ alkoxy, and C₁₋₆ haloalkoxy.

3. The compound of claim 1 or a pharmaceutically acceptable salt thereof, wherein R¹ is phenyl optionally substituted with one or more substituents each independently selected from the group consisting of halogen, cyano, and C₁₋₆ haloalkoxy.

4. The compound of claim 1 or a pharmaceutically acceptable salt thereof, wherein R¹ is phenyl substituted with cyano, phenyl substituted with halogen or phenyl substituted with two halogens.

5-6. (canceled)

7. The compound of claim 1 or a pharmaceutically acceptable salt thereof, wherein R² is phenyl optionally substituted with one or more substituents each independently selected from C₁₋₆ alkyl and halogen.

8. The compound of claim 1, wherein the compound is represented by formula (Ia) 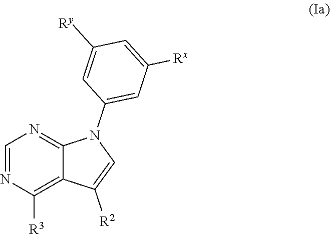
or a pharmaceutically acceptable salt thereof, wherein
R^{x} is selected from the group consisting of halogen, cyano, and C₁₋₆ haloalkoxy;
R^{y} is selected from hydrogen and halogen;
R² is phenyl optionally substituted with one or more C₁₋₆ alkyl or halogen;
R³ is 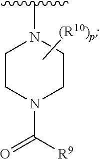
each R¹⁰ is independently selected from C₁₋₆ alkyl and cycloalkyl;
R⁹ is C₁₋₆ alkyl substituted with one or more hydroxy or C₁₋₆ alkoxy and optionally substituted with one or more substituents each independently selected from halogen, C₁₋₃ haloalkyl, and C₁₋₆ haloalkoxy; and
p is 0, 1, 2, 3, or 4.

9. The compound of claim 8 or a pharmaceutically acceptable salt thereof, wherein R^{x} is cyano, fluoro or chloro.

10. (canceled)

11. The compound of claim 8 or a pharmaceutically acceptable salt thereof, wherein R^{y} is hydrogen or fluoro.

12. (canceled)

13. The compound of claim 1 or a pharmaceutically acceptable salt thereof, wherein R² is phenyl optionally substituted with halogen.

14. The compound of claim 1 or a pharmaceutically acceptable salt thereof, wherein p is 2.

15. The compound of claim 1 or a pharmaceutically acceptable salt thereof, wherein R³ is

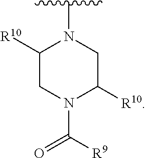

16. The compound of claim 1 or a pharmaceutically acceptable salt thereof, wherein each R¹⁰ is independently C₁₋₆ alkyl.

17. The compound of claim 15 or a pharmaceutically acceptable salt thereof, wherein R³ is

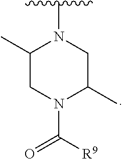

18. (canceled)

19. The compound of claim 1 or a pharmaceutically acceptable salt thereof, wherein R⁹ is C₁₋₆ alkyl substituted with one or more methoxy or hydroxy.

20. The compound of claim 19 or a pharmaceutically acceptable salt thereof, wherein R⁹ is selected from the group consisting of

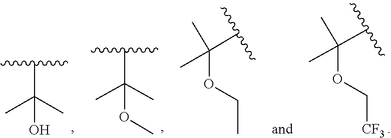

21. A compound selected from the group consisting of: 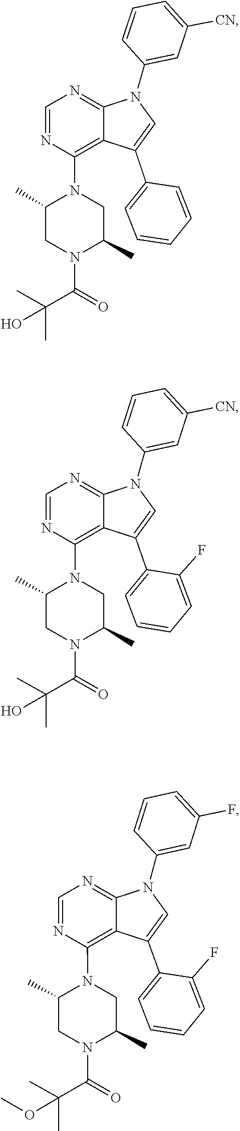 [SMILES: C[C@@H]1CN(c2ncnc3c2c(-c2ccccc2)cn3-c2cccc(C#N)c2)[C@@H](C)CN1C(=O)C(C)(C)O ; C[C@@H]1CN(c2ncnc3c2c(-c2ccccc2F)cn3-c2cccc(C#N)c2)[C@@H](C)CN1C(=O)C(C)(C)O ; COC(C)(C)C(=O)N1C[C@H](C)N(c2ncnc3c2c(-c2ccccc2F)cn3-c2cccc(F)c2)C[C@H]1C] 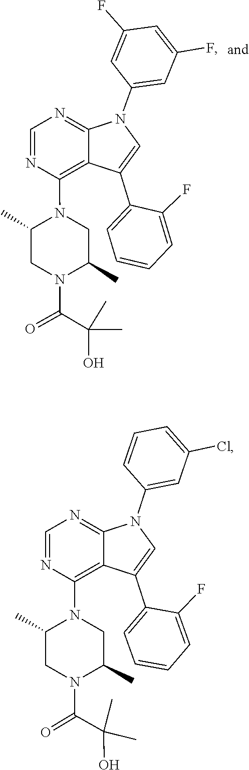 [SMILES: C[C@@H]1CN(c2ncnc3c2c(-c2ccccc2F)cn3-c2cc(F)cc(F)c2)[C@@H](C)CN1C(=O)C(C)(C)O ; C[C@@H]1CN(c2ncnc3c2c(-c2ccccc2F)cn3-c2cccc(Cl)c2)[C@@H](C)CN1C(=O)C(C)(C)O]
or a pharmaceutically acceptable salt thereof.

22. A pharmaceutical composition comprising a pharmaceutically acceptable excipient and the compound of claim 1 or a pharmaceutically acceptable salt thereof.

23. A method of(a modulating TRPMLs, (b) treating a disorder which can be treated by modulation of TRPMLs, (c) treating a disorder which can be treated by modulation of lysosomes, or (d) treating a disorder selected from the group consisting of a ciliopathy, neurodegenerative disorder, lysosomal storage disorder, lysosomal transport disorder, glycogen storage disorder, cholesteryl ester storage disease, a muscular disease, a disease related to aging, macular degeneration and cancer, the method comprising administering to a patient in need thereof a therapeutically effective amount of the compound of claim 1 or a pharmaceutically acceptable salt thereof.

24-27. (canceled)

28. A method of treating a disorder, the method comprising administering to a patient in need thereof a therapeutically effective amount of the compound of claim 1, or a pharmaceutically acceptable salt thereof, wherein:
a) the disorder is a ciliopathy and the ciliopathy is selected from the group consisting of polycystic kidney disease, pancreatic cysts in polycystic kidney disease, Bardet-Biedl syndrome, nephronophthisis, Joubert Syndrome, Mecke-Gruber Syndrome, oral-facial-digital syndrome, Senior Loken Syndrome, Birt-Hogg-Dube syndrome, Leber's congenital amaurosis, Alstrom syndrome, Jeune asphyxiating thoracic dystrophy, Ellis van Creveld syndrome, Sensenbrenner syndrome, and primary ciliary dyskinesia;
(b) the disorder is a polycystic kidney disease and the polycystic kidney disease is autosomal dominant polycystic kidney disease, autosomal recessive polycystic kidney disease, or pancreatic cysts associated with autosomal dominant polycystic kidney disease;
(c) the disorder is a neurodegenerative disorder and the neurodegenerative disorder is selected from the group consisting of Parkinson's disease, GBA-Parkinson's disease, LRRK2 Parkinson's disease, Huntington's disease, amyotrophic lateral sclerosis (ALS), Alzheimer's disease, progressive supranuclear palsy, frontotemporal dementia, FTDP-17, corticobasal degeneration, Lewy body dementia, Pick's disease, and multi system atrophy;
(d) the disorder is a lysosomal storage disorder and the lysosomal storage disorder is selected from the group consisting of Niemann-Pick disease, Gaucher's disease, neuronopathic Gaucher's disease, sphingolipidoses, Farber disease, Krabbe disease, Galactosialidosis, gangliosidoses, Gaucher Disease, Lysosomal acid lipase deficiency, sulfatidoses, mucopolysaccharidoses, mucolipidoses, lipidoses, and oligosaccharidosis;
(e) the disorder is a lysosomal storage disorder and the lysosomal storage disorder is selected from the group consisting of sphingolipidoses, Farber disease, Krabbe disease, Galactosialidosis, Fabry disease, Schindler disease, beta-galactosidase disorder, GM1 gangliosidosis, GM2 gangliosidosis AB variant, GM2 gangliosidosis activator deficiency, Sandhoff disease, Tay-Sachs disease, Gaucher disease, lysosomal acid lipase deficiency, Niemann-Pick disease, metachromatic leukodystrophy, Saposin B deficiency, multiple sulfatase deficiency, Hurler syndrome, Scheie sundrome, Hurler-Scheie syndrome, Hunter syndrome, Sanfilippo syndrome, Morquio syndrome, Maroteaux-Lamy syndrome, Sly syndrome, hyaluronidase deficiency, sialidosis, I-cell disease, pseudo-Hurler polydystrophy, phosphotransferase deficiency, mucolipin 1 deficiency, Santavuori-Haltia disease, Jansky-Bielchowsky disease, Batten-Spielmeyer-Vogt disease, Kufs disease, Finnish variant neuronal ceroid lipfuscinosis, late infantile variant neuronal ceroid lipfuscinosis, type 7 neuronal ceroid lipfuscinosis, northern epilepsy neuronal ceroid lipfuscinosis, Turkish late infantile neuronal ceroid lipfuscinosis, German/Serbian late infantile neuronal ceroid lipfuscinosis, congenital cathepsin D deficiency, Wolman disease, alpha-mannosidosis, beta-mannosidosis, aspartylgluosaminuria, and fucosidosis;
(f) the disorder is a lysosomal transport disease selected from the group consisting of cystinosis, pycnodysostosis, Salla disease, sialic acid storage disease, and infantile free sialic acid storage disease; or
(g) the disorder is a glycogen storage disease selected from the group consisting of Pompe disease and Danon disease.

29-39. (canceled)
